(19) United States
(12) Patent Application Publication
(10) Pub. No.: US 2022/0282222 A1
(43) Pub. Date: Sep. 8, 2022

(54) BIO-PRINTED KIDNEY TISSUE

(71) Applicant: MURDOCH CHILDREN'S RESEARCH INSTITUTE, Parkville, Victoria (AU)

(72) Inventors: Melissa H. LITTLE, Parkville, Victoria (AU); Kynan LAWLOR, Parkville, Victoria (AU); Jessica VANSLAMBROUCK, Parkville, Victoria (AU); Sean WILSON, Parkville, Victoria (AU)

(21) Appl. No.: 17/636,422

(22) PCT Filed: Aug. 21, 2020
(86) PCT No.: PCT/AU2020/050882

§ 371 Date: Feb. 18, 2022

(30) Foreign Application Priority Data
Aug. 23, 2019 (AU) 2019903094

(51) Int. Cl. C12N 5/071; A61L 27/36; A61L 27/38

(52) U.S. Cl. CPC C12N 5/0686; A61L 27/3641; A61L 27/3804; C12N 5/0697; C12N 2513/00; C12N 2501/119; C12N 2501/415

(57) ABSTRACT

The present disclosure relates to bio-printed kidney tissue and methods of manufacturing the same. The bio-printed tissue and methods may be used in a variety of applications such as regenerative medicine.

### FIELD

The present disclosure relates to bio-printed kidney tissue and methods of manufacturing the same. The bio-printed tissue and methods may be used in a variety of applications such as disease modelling, drug screening, drug testing, renal replacement, tissue engineering and regenerative medicine.

### CROSS REFERENCE TO RELATED APPLICATIONS

The present application claims the benefit of priority to Australian provisional application No. 2019903094, filed on 23 Aug. 2019, the entire contents of which is herein incorporated by reference.

### BACKGROUND

Kidneys play a major role in removal of waste products and maintain body fluid volume. The functional working units of the kidney are known as nephrons. The human kidneys contain up to 2 million epithelial nephrons responsible for blood filtration, all of which arise from nephron progenitors before birth. No nephron progenitors exist in the postnatal human kidney. This absence of a nephron progenitor population ensures no ability for new nephron formation (neo-nephrogenesis) and therefore, subsequent injury, aging and disease can lead to reduced nephron number and consequential chronic kidney disease (CKD). This eventually results in end stage kidney disease (ESKD) which is incompatible with life unless treated with some form of renal replacement, including either dialysis (peritoneal dialysis or haemodialysis) or organ transplantation. These are the only available treatment options for ESKD. Both dialysis and kidney transplantation are costly, have significant disadvantages and affect the quality of life of the patient. At present, with CKD increasing at 6% pa worldwide and only 1 in 4 patients able to access a donor organ, a source of replacement kidney tissue is a major therapeutic target.

The directed differentiation of human pluripotent stem cells (hPSCs), including both human embryonic stem cells (hES) and human induced pluripotent stem cells (hiPS), to distinct cellular endpoints has enabled the generation of organoid models of a variety of human tissues, including the kidney. Previous organoid models such as those discussed in Takasato et al. (2015) Nature, Vol. 526:564-568 may produce kidney organoids having a complex three-dimensional structure, which are multicellular models of the human kidney. These complex, multicellular structures contain fully segmented nephrons associated with a collecting duct network surrounded by renal interstitium and endothelial cells. They show gene expression equivalent to the human fetal kidney in Trimester 1 of development.

Despite this important outcome, kidney organoids produced according to previously described methods are self-limiting due to the lack of a sustained nephron progenitor population. During normal human kidney development, ongoing nephron formation occurs from a persisting nephron progenitor population. While it has been shown that this population is present in a kidney organoid, it has also been shown that this population of progenitors generates nephrons and is then lost, limiting the maximal nephron number that can be generated using this approach (Howden et al, EMBO Reports, 2019). A key factor for the generation of maximal functional kidney tissue from stem cells is the relative proportion of the tissue comprised of nephrons. A second key factor is the reduction of unwanted non-renal populations. Accordingly, engineered kidney tissue with a greater number of nephrons per number of cells used to generate such tissue and with more uniform distribution of nephrons is required. A third key factor is kidney tissue in which the component nephrons showed improved patterning and evidence of nephron segment maturation. A fourth key factor to the generation of transplantable renal replacement tissue is a capacity to manufacture such tissue in a reliable and reproducible fashion amenable to automation.

### SUMMARY OF INVENTION

The inventors have surprisingly found that an in-vitro engineered, or bio-printed, kidney tissue, derived from a composition comprising stem cell-derived renal progenitor cells, can be generated with an increased or decreased number of nephrons arising per number of cells used to generate the tissue, depending on the spatial parameters applied to the bio-printing of the tissue. It has also been surprisingly found that it is possible to generate bio-printed tissue with more uniform distribution of nephrons. The inventors have surprisingly discovered that by modifying the spatial parameters of bio-printed tissue, more nephrons can be generated from the same amount of starting material (cells), and that the resulting tissue has improved characteristics. Accordingly, described herein are bio-printed kidney tissues enriched for maturing nephrons and methods for producing the same.

According to a first aspect, the present invention provides bio-printed kidney tissue, wherein the bio-printed kidney tissue is enriched with nephrons which are distributed throughout the tissue. In a preferred embodiment, the nephrons are evenly or uniformly distributed through the printed tissue.

According to a second aspect, the present invention provides bio-printed kidney tissue comprising a predetermined amount of a bio-ink, wherein the bio-ink comprises a plurality of cells, wherein the bio-ink is bio-printed in a layer that is less than about 50 μm high and wherein the bio-printed bio-ink is induced to form kidney tissue. In another embodiment, the height of the bio-printed kidney tissue after the bio-printed bio-ink is induced to form kidney tissue is about 150 μm or less. In other words, the height of the final bio-printed kidney tissue is about 150 μm or less.

According to a third aspect, the present invention provides bio-printed kidney tissue comprising a predetermined amount of a bio-ink, wherein the bio-ink comprises a plurality of cells and the bio-ink is bio-printed in a layer that comprises about 30,000 cells per mm² or less.

According to a fourth aspect, the present invention provides a method for producing bio-printed kidney tissue comprising the steps of: bio-printing a pre-determined amount of a bio-ink onto a surface, wherein the bio-ink comprises a plurality of cells, and wherein the bio-ink is bio-printed in a layer that is less than about 50 μm high; and inducing the bio-printed, pre-determined amount of the bio-ink to form bio-printed kidney tissue.

According to a fifth aspect, the present invention provides a method for producing bio-printed kidney tissue comprising the steps of: bio-printing a pre-determined amount of a bio-ink onto a surface, wherein the bio-ink comprises a plurality of cells that are bio-printed in a layer that comprises about 30,000 cells per mm² or less; and inducing the bio-printed, pre-determined amount of the bio-ink to form bio-printed kidney tissue.

According to a sixth aspect, the present invention provides bio-printed kidney tissue produced according to the method of the fourth or fifth aspect.

According to a seventh aspect, the present invention provides bio-printed kidney tissue of any one of the first, second, third or sixth aspects, for use in the treatment of kidney disease or renal failure in a subject in need thereof.

According to an eighth aspect, the present invention provides use of bio-printed kidney tissue of any one of the first, second, third or sixth aspects, in the manufacture of a medicament for the treatment of kidney disease in a subject in need thereof.

According to a ninth aspect, the present invention provides a method of treating kidney disease or renal failure in a subject in thereof, comprising administering to the subject bio-printed kidney tissue of any one of the first, second, third or sixth aspects.

Numbered statements of the invention are as follows:

1. Bio-printed kidney tissue, wherein the bio-printed kidney tissue is enriched with nephrons which are distributed throughout the tissue.

2. The bio-printed kidney tissue of statement 1, wherein the bio-printed kidney tissue is a layer of bio-printed tissue comprising a surface area of nephron tissue of greater than 0.2 mm² per 10,000 cells printed.

3. The bio-printed kidney tissue of statement 1 or 2, wherein the bio-printed kidney tissue is a layer of bio-printed kidney tissue comprising about 30,000 cells per mm² or less when printed.

4. The bio-printed kidney tissue of any one of the preceding statements, wherein the bio-printed kidney tissue expresses high levels of any one or more of SULT1E1, SLC30A1, SLC51B, FABP3, HNF4A, CUBN, LRP2, EPCAM and MAFB.

5. The bio-printed kidney tissue of statement 4, wherein the bio-printed kidney tissue comprises nephrons in which the proximal tubule and distal tubule segments express markers of maturation, including HNF4A and SLC12A1.

6. The bio-printed kidney tissue of statement 4 or 5, wherein the bio-printed kidney tissue expresses each of the markers HNF4A, CUBN, LRP2, EPCAM and MAFB.

7. The bio-printed kidney tissue of any one of the preceding statements, wherein the height of the bio-printed kidney tissue is about 50 μm or less when printed.

8. The bio-printed kidney tissue of any one of the preceding statements, wherein the bio-printed kidney tissue has a length of from about 1 mm to about 30 mm and a width of from about 0.5 mm to about 20 mm.

9. The bio-printed kidney tissue of statement 7 or 8, wherein the bio-printed kidney tissue comprises from about 5 to about 100 nephrons/mm² of bio-printed kidney tissue.

10. Bio-printed kidney tissue comprising a predetermined amount of a bio-ink, wherein the bio-ink comprises a plurality of cells, wherein the bio-ink is bio-printed in a layer that is about 50 μm high or less and wherein the bio-printed bio-ink is induced to form kidney tissue.

11. The bio-printed kidney tissue of statement 10, wherein the bio-ink is bio-printed in a layer selected from about 20 μm high to about 40 μm high.

12. The bio-printed kidney tissue of statement 10, wherein the bio-ink is bio-printed in a layer about 30 μm high.

13. The bio-printed kidney tissue of statement 10, wherein the bio-ink is bio-printed in a layer about 25 μm high.

14. The bio-printed kidney tissue of any one of statements 10-14, wherein the predetermined amount of bio-ink comprises between approximately 10,000 cells/μl and approximately 400,000 cells/μl.

15. The bio-printed kidney tissue of any one of statements 10-14, wherein said plurality of cells comprises partly differentiated cells.

16. The bio-printed kidney tissue of any one of statements 10-15, wherein said plurality of cells comprises renal progenitor cells.

17. The bio-printed kidney tissue of statement 16, wherein the renal progenitor cells comprise nephron progenitor cells.

18. The bio-printed kidney tissue of statement 16 or 17, wherein the renal progenitor cells comprise ureteric epithelial progenitor cells.

19. The bio-printed kidney tissue of any one of statements 10-15, wherein said plurality of cells comprises intermediate mesoderm cells.

20. The bio-printed kidney tissue of any one of statements 10-15, wherein said plurality of cells comprises metanephric mesenchyme cells.

21. The bio-printed kidney tissue of any one of statements 10-15, wherein said plurality of cells comprises nephric duct cells.

22. The bio-printed kidney tissue of any one of statements 10-15, wherein said plurality of cells comprises fully differentiated cells.

23. The bio-printed kidney tissue of any one of statements 10-22, wherein said plurality of cells comprises patient-derived cells.

24. The bio-printed kidney tissue of any one of statements 10-23, wherein said plurality of cells comprises cells from a reporter cell line.

25. The bio-printed kidney tissue of any one of statements 10-24, wherein said plurality of cells comprises gene-edited cells.

26. The bio-printed kidney tissue of any one of statements 10-25, wherein said plurality of cells comprises diseased cells, healthy cells, or a combination of diseased and healthy cells.

27. The bio-printed kidney tissue of any one of statements 10-26, wherein the bio-printed kidney tissue comprises a surface area of nephron tissue of greater than 0.2 mm² per 10,000 cells printed.

28. The bio-printed kidney tissue of any one of statements 10-27, wherein the bio-printed kidney tissue comprises about 30,000 cells per mm² or less when printed.

29. The bio-printed kidney tissue of any one of statements 10-28, wherein the bio-printed kidney tissue expresses high levels of any one or more of HNF4A, CUBN, LRP2, EPCAM and MAFB.

30. The bio-printed kidney tissue of statement 29, wherein the bio-printed kidney tissue comprises nephrons in which the proximal tubule and distal tubule segments express markers of maturation, including HNF4A.

31. The bio-printed kidney tissue of statement 29 or 30, wherein the bio-printed kidney tissue expresses each of the markers HNF4A, CUBN, LRP2, EPCAM and MAFB.

32. The bio-printed kidney tissue of any one of statements 1-31, wherein the tissue comprises from about 5 to about 100 nephrons/10,000 cells printed.

33. The bio-printed kidney tissue of any one of statements 10-32, wherein the tissue has an even distribution of nephrons throughout the bio-printed layer.

34. The bio-printed kidney tissue of any one of statements 10-33, wherein the tissue has an even distribution of glomerular structures expressing MAFB throughout the bio-printed layer.

35. The bio-printed kidney tissue of any one of statements 1-34, further comprising a bio-compatible scaffold.

36. The bio-printed kidney tissue of statement 35, wherein bio-ink is bio-printed onto a bio-compatible scaffold.

37. The bio-printed kidney tissue of any one of statements 35 or 36, wherein the biocompatible scaffold is a hydrogel.

38. The bio-printed kidney tissue of any one of statements 35-37, wherein the biocompatible scaffold is biodegradable or bio-absorbable.

39. The bio-printed kidney tissue of any one of statements 10-38, wherein the bio-ink further comprises one or more bioactive agents.

40. The bio-printed kidney tissue of statement 39, wherein said one or more bioactive agents promotes induction of kidney tissue from said plurality of cells.

41. A method for producing bio-printed kidney tissue comprising the steps of: bio-printing a pre-determined amount of a bio-ink onto a surface, wherein the bio-ink comprises a plurality of cells, and wherein the bio-ink is bio-printed in a layer that is about 50 μm high or less; and inducing the bio-printed, pre-determined amount of the bio-ink to form bio-printed kidney tissue.

42. The method of statement 41, wherein at the step of bio-printing the bio-ink is bio-printed in a layer selected from about 20 μm high to about 40 μm high.

43. The method of statement 41, wherein at the step of bio-printing, wherein the bio-ink is bio-printed in a layer about 30 μm high.

44. The method of statement 41, wherein at the step of bio-printing the bio-ink is bio-printed in a layer about 25 μm high.

45. The method of any one of statements 41-44, wherein the predetermined amount of bio-ink comprises between approximately 10,000 cells/μl and approximately 400,000 cells/μl.

46. The method according to statement 45, wherein the bio-ink comprises about 200,000 cells/μl.

47. The method of any one of statements 41-46, wherein said plurality of cells comprises partly differentiated cells.

48. The method of any one of statements 41-46, wherein said plurality of cells comprises renal progenitor cells.

49. The bio-printed kidney tissue of statement 48, wherein the renal progenitor cells comprise nephron progenitor cells.

50. The method of statement 48 or 49, wherein the renal progenitor cells comprise ureteric epithelial progenitor cells.

51. The method of any one of statements 41-47, wherein said plurality of cells comprises intermediate mesoderm cells, preferably a culture expanded population of stem cell-derived intermediate mesoderm cells.

52. The method of any one of statements 41-47, wherein said plurality of cells comprises metanephric mesenchyme cells.

53. The method of any one of statements 41-47, wherein said plurality of cells comprises nephric duct cells.

54. The method of any one of statements 41-47, wherein said plurality of cells comprises fully differentiated cells.

55. The method of any one of statements 41-54, wherein said plurality of cells comprises patient-derived cells.

56. The method of any one of statements 41-55, wherein said plurality of cells comprises cells from a reporter cell line.

57. The method of any one of statements 41-56, wherein said plurality of cells comprises gene-edited cells.

58. The method of any one of statements 41-57, wherein said plurality of cells comprises diseased cells, healthy cells, or a combination of diseased and healthy cells.

59. The method of any one of statements 41-58, wherein the bio-printed kidney tissue comprises from about 5 to about 100 nephrons/10,000 cells printed.

60. The method of any one of statements 41-59, wherein the bio-printed kidney tissue has an even distribution of nephrons throughout the bio-printed layer.

61. The method of any one of statements 41-60, wherein the bio-printed kidney tissue has an even distribution of glomerular structures expressing MAFB throughout the bio-printed layer.

62. The method of any one of statements 41-61, wherein at the step of bio-printing the bio-ink is bio-printed onto a bio-compatible scaffold.

63. The method of statement 62, wherein the biocompatible scaffold is a hydrogel.

64. The method of any one of statements 62 or 63, wherein the biocompatible scaffold is biodegradable or bio-absorbable.

65. The method of any one of statements 41-64, wherein the bio-ink further comprises one or more bioactive agents.

66. The method of statement 65, wherein said one or more bioactive agents promotes induction of kidney tissue from said plurality of cells.

67. The method of any one of statements 41-66, wherein the step of inducing comprises contacting the bio-printed, predetermined amount of bio-ink with FGF-9.

68. The method of statement 67, wherein the step of inducing comprises contacting the bio-printed, predetermined amount of bio-ink with FGF-9 for a period of 5 days.

69. The method of any one of statements 41-68, wherein the plurality of cells is contacted with a cell culture medium comprising CHIR before being bio-printed.

70. The method of any one of statements 41-69, wherein the bio-printing step uses an extrusion-based bio-printer.

71. The method of any one of statements 41-70, wherein at the step of bio-printing, a dispensing apparatus of a bio-printer is configured to dispense said layer in one or more lines.

72. The method of any one of statements 41-71, wherein at the step of bio-printing, a dispensing apparatus of a bio-printer is configured to dispense said layer in one or more lines so as to form a continuous sheet or patch.

73. Bio-printed kidney tissue produced according to any one of statements 41-72.

74. Bio-printed kidney tissue of any one of statements 1-40, or 73, for use in the treatment of kidney disease or renal failure in a subject in need thereof.

75. Use of bio-printed kidney tissue of any one of statements 1-40, or 73, in the manufacture of a medicament for the treatment of kidney disease in a subject in need thereof.

76. A method of treating kidney disease or renal failure in a subject in thereof, comprising administering to the subject bio-printed kidney tissue of any one of statements 1-40, or 73.

77. The bio-printed kidney tissue of any one of statements 1-40, or 73, for use according to statement 74, the use of statement 75, or the method of statement 76, wherein in said treatment the bio-printed kidney tissue is transplanted under the renal capsule of said subject.

Any example or embodiment herein shall be taken to apply mutatis mutandis to any other example or embodiment unless specifically stated otherwise.

The present disclosure is not to be limited in scope by the specific examples described herein, which are intended for the purpose of exemplification only. Functionally equivalent products, compositions and methods are clearly within the scope of the disclosure, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The disclosure is hereinafter described by way of the following non-limiting Examples and with reference to the accompanying drawings.

### BRIEF DESCRIPTION OF DRAWINGS

FIG. 1. Generation of highly reproducible human pluripotent stem cell-derived kidney organoids via extrusion-based cellular bio-printing of day 7 intermediate mesoderm cell paste. A. Protocol for differentiating pluripotent stem cells and bio-printing to generate kidney organoids. This diagram illustrates the point at which bio-printing is used to replace manual handling and compares the relative cell count and speed of organoid generation between manual handling (Takasato et al., 2016) and 3D cell paste extrusion bio-printing. B. Brightfield images of micromass cell paste cultures from day of printing (day 7+0) to day 20 of culture (day 7+20) showing the spontaneous formation of nephrons across time. C. Wholemount immunofluorescence staining of day 7+18 organoid showing evidence for patterned and segmented nephrons including distal tubule (E-CADHERIN, green), proximal tubule (LTL, blue), podocytes (NEPHRIN, white) and connecting segment/collecting duct (GATA3, red). Merge of channels illustrates the relationship of individual nephron segments. D. Wholemount immunofluorescence staining of day 7+18 bio-printed organoids with markers illustrating the presence of proximal tubular segments (CD13, CUBN, LTL), tubular basement membranes (LAMININ), surrounding stroma (MEIS1/2), distal tubule/loop of Henle TAL (SLC12A1) and endothelium (CD31). E. Brightfield (day 7+7) and wholemount immunofluorescence-stained manual and bio-printed kidney organoids generated simultaneously from the same batch of iPSC-derived intermediate mesoderm. Staining shows evidence of patterning and segmented nephrons in both manual and bio-printed organoids (EPCAM, green: epithelium; LTL, blue: proximal tubule; NPHS1, white: glomeruli; GATA3, red: connecting segment/collecting duct). F. Transwell® inserts onto which triplicate kidney organoids have been bio-printed. The starting cell number is indicated. The top row illustrates a capacity to generate organoids with reducing numbers of cells. The bottom row illustrates the reproducibility of size when bio-printing a given cell number across multiple wells. G. 6-well Transwell® insert with 9 bio-printed organoids, each containing approximately 96,000 cells. H. Kidney organoid differentiation within bio-printed organoids is equivalent with reduced starting cell number. Images show H&E stained sections from mature organoids printed as either 2×10⁵ or 4×10⁵ cell organoids.

FIG. 2. A. Histological cross section of an entire day 7+18 bio-printed kidney organoid showing clear evidence of an interconnecting epithelium (arrowheads) from which nephrons arise. B. Immunostaining of a bio-printed kidney organoid section showing a GATA3+ECAD+ connecting segment/collecting duct with multiple attached ECAD+GATA3-nephrons. C. Immunostaining of bio-printed kidney organoid section showing ECAD+ nephrons attached to MAFB+ glomeruli. D. Brightfield, histological and immunofluorescence comparisons of kidney organoids generated manually (5×10⁵ cells per organoid), using dry cell paste controlled for organoid diameter versus dry cell paste controlled for cell number versus wet cell paste.

FIG. 3. A. Immunofluorescence of organoids from a single starting differentiation used to generate manual organoids (5×10⁵ cells) versus bio-printed organoids generated from as few as 4,000 cells. B. Differentiation time course of bio-printed organoids generated using the MAFB^{mTagBFP2} reporter iPSC line. C. MAFB^{mTagBFP2} bio-printed organoids on the same Transwell filter with 4K, 50K or 100K of cells per organoid showing fluorescence reporter imaging (blue) and staining for differentiation (ECAD, green; LTL, blue; GATA3, red; NPHS1, purple). D. MAFB^{mTagBFP2} bio-printed organoids on the same Transwell filter all generated using 100K of cells per organoid showing live fluorescence imaging (blue) and staining for differentiation (ECAD, green; LTL, blue; GATA3, red; NPHS1, purple).

FIG. 4. Application of bio-printed organoids for compound screening in 96-well format. A. Image of all bio-printed organoids within a 96-well Transwell format. Bio-printed organoids were generated using a deposition of 1×10⁵ cells per organoid and cultured for a further 18 days. B. 96-well plate, secured onto the print stage within a plate holder just prior to deposition C. Quality control assessment of bio-printed cell number per organoid and cell viability across a 96-well plate. D. Immunofluorescence analysis of response to Doxorubicin at 10.0 uM versus control. Sections of bio-printed organoids were stained with antibodies to MAFB (podocyte marker), cleaved caspase 3 (CC3; apoptotic marker), cytokeratin 8/18 (CCK8/18, tubule marker), lotus tetranoglobulus lectin (LTL, proximal tubule) and DAPI to mark nuclei. The podocyte specific loss of MAFB expression and induction of apoptosis was seen in the presence of doxorubicin. E. Gene expression of kidney injury molecule-1 (HAVCR) and apoptosis genes (CASP3, BAX) in response to Doxorubicin treatment. F. Gene expression of key podocyte (NPHS1, PODXL) and proximal tubule (CUBN) genes in response to Doxorubicin treatment. G. Evaluation of cell viability in response to Doxorubicin treatment comparing data from bio-printed organoids deposited in 6-well (green) and 96-well (blue) Transwell format. Viability was assessed at 72 hours after addition of Doxorubicin. H. Application of 96-well bio-printed organoids for screening viability in response to a series of aminoglycoside antibiotics.

FIG. 5. Use of extrusion bio-printing to alter organoid conformation. A. Generation of a series of organoids of increasing length from an identical starting cell number (1.1×10⁵ cells). The diagram serves to illustrate the relative effect on organoid profile/height at bio-printing, moving from ratio 0 (no needle movement at extrusion) to ratio 40 (extrusion with needle movement across the Transwell surface), not to scale. Ratio refers to the ratio of tip movement to extrusion B. Fluorescent beads were included to measure cell paste spreading across the Transwell surface area as organoids were being produced. More spreading results in less beads per surface area. Representative regions are shown from ratio 0 and 40 cell paste deposition. White dotted lines mark the edge of cell paste. C. Quantification of beads density per unit of Transwell surface area. Higher ratios give more spreading and hence lower beads densities (n=21 organoids total, n=3 per condition except for ratio 0 where n=9). D. Measured tissue height at D7+0, shortly after bio-printing (n=27 organoids from 2 independent experiments). E. Measured organoid height at day 7+12 for organoids printed with varying conformations (n=21 organoids). Red points in D and E represent mean value. Note that the Y-axis scale differs between D and E. See FIG. 6 for further detail. F. Representative fluorescent imaging of live organoids generated using the MAFB^{mTGABFP2} reporter line with blue fluorescent protein marking glomerular area in organoids printed in varying conformations. G. Quantification of mTagBFP2 area versus measured organoid length in replicate bio-printed organoids of different conformations. Each point represents a single organoid (n=90 organoids total, see FIG. 6). H. Immunofluorescence of representative bio-printed organoids from each conformation showing MAFB^{mTagBFP2} (glomeruli, blue endogenous fluorescence), epithelium (EPCAM, grey), proximal tubule (LTL, green) and connecting segment/collecting duct (GATA3, red).

FIG. 6. Quantification of bead density and MAFB^{mTagBFP2} reporter signal in organoids with varied conformations. A. Representative image of fluorescent bead signal (greyscale) at D7+0 across an entire print pattern showing all 5 conformations, from left to right: ratio 0 (3 replicates), ratio 40, ratio 30, ratio 20, ratio 10. B. Composite image of each conformation at D7+12 showing mTagBFP2 reporter expression (cyan) and bead signal (red). Note images are placed on a black background. Scale bar is 1 mm for A and B. C. Quantification of total organoid area (refer to Methods) and mTagBFP2 area in replicate organoids (compare to FIG. 7G). D. Table of organoid numbers by replicate plate and ratio used for quantification in C and FIG. 7G. E. Example of 9 replicate organoids produced using ratio 20. Organoids are consistent between 3 organoids from separate wells on each plate, and between plates. F. Representative images of sparse labelling with CellTrace Far Red dye to quantify organoid height at D7+0. XY and orthogonal view are shown. G. Schematic of the scoring method used for quantification.

FIG. 7. Changing organoid conformation reduces unpatterned tissue and increases nephron number and maturation (also refer to FIG. 9). A. Heatmap comparing scaled log counts per million expression values in bulk-RNAseq transcriptional profiles of ratio 0 (R0), ratio 20 (R20) and ratio 40 (R40) organoids. B. Heatmap of scaled log counts per million expression values of genes representing the top most significantly enriched GO terms in ratio 40 vs ratio 0 organoids. C. Immunofluorescence to validate transcriptional changes, illustrating a reduction in the endothelial marker SOX17 and an increase in the loop of Henle thick ascending limb (TAL) marker SLC12A1 as ratio increases. D. 3D rendering of bio-printed organoids illustrating the distinct morphology between a ratio 0 and a ratio 40 organoid. Images are rendered to show the XY plane tilted at 45 degrees.

FIG. 8. Single cell RNAseq comparison of manual organoids, bio-printed R0 ‘dots’ and bio-printed R40 ‘lines’. A. Experimental design. Multiple organoid sets were generated per conformation, and each set is barcoded then combined to form a single scRNAseq library per condition. Both bio-printed types are generated from 1.1×10⁵ cells, while manual organoids are generated from 2.3×10⁵ cells. B. Image quantification confirms an increase in nephrons in R40 line organoids based on MAFB reporter area. Black bars represent the mean value. R40-Man, p=2.1×10⁻⁵, R40-R0, p=2×10⁻¹⁶ based on pairwise t-tests with the Holm multiple comparison correction. Details of n values per condition, set level comparisons and representative images are in FIG. 9. C. UMAP visualising transcriptional variation in stromal lineage cells in scRNA. See FIG. 10 for further details of cluster identity. D. Proportion of each stromal cell cluster by replicate and condition. P-value is stated where p<0.2 and represents one-way ANOVA comparing all 3 conditions. Each point represents a single replicate while red diamonds represent mean values for n=4. E. UMAP visualising transcriptional variation in nephron lineage cells in scRNAseq data. Clusters are Nephron Progenitor-like (3), Pre-podocyte (4), Podocyte (1), Pre-Tubule (2), Distal Tubule (0) and Proximal Tubule (8). Cluster 5 and 7 represent cycling cells and cluster 6 was removed as it represented doublet cells. See FIG. 10 for further details. F. Proportions of each nephron cell type per replicate across conditions. P-value is stated where p<0.2 and represents one-way ANOVA comparing all 3 conditions. For cluster 4 ANOVA was followed by a Tukey multiple comparison of means, giving p=0.021 for R40 vs Man. G. Heatmap indicating the number of filtered differentially expressed (DE) genes within each cluster between conformations for nephron lineage clusters. DE testing is conducted on summed pseudo-bulk counts for a given cell type per replicate and condition and takes into account variability between replicates (n=4) to identify changes that are statistically significant (adjusted p-value <0.05). Gene lists were filtered to remove genes appearing in more than 3 cell types, thus focusing on specific changes and minimising possible batch effects. H. Violin plots of normalised single cell expression values for selected genes identified as having statistically significant DE between R40 and Manual organoids in pseudo-bulk analysis of proximal tubule cells (nephron cluster 8). Violin plots show the distribution of single cell expression values as a coloured shape, with individual points overlayed as black dots. R40 organoids show increased expression of genes associated with proximal tubule maturity (SLC30A1, SLC51B, FABP3, SULT1E1) and decreased expression of genes associated with early immature tubule (SPP1, JAG1) compared to manual organoids. I. Heatmap indicating the number of filtered differentially expressed genes within each cluster between conformations for stromal lineage clusters. J. Violin plots of normalised single cell expression values for selected genes identified as having significantly increased expression in pseudo-bulk analysis of stromal cluster 2 cells between R40 and Manual organoids. K,L. Violin plots of normalised expression values for selected genes with significantly increased expression in pseudo-bulk analysis of stromal cluster 3 cells in R40 organoids. Genes associated with nephron progenitor identity were significantly increased in K) R40 vs Manual organoids (HOXA11, FOXC2) and in L) R40 vs R0 organoids (EYA1, SIX1).

FIG. 9. Quantification of large image data sets associated with organoids used for single cell RNA seq. Line organoids are approximately 12 mm long. A. Representative images from 3 separate wells across replicates and conditions. B. Quantification of MAFB-mTagBFP2 reporter area by set and condition. Data is as in FIG. 8B, but here is separated by set. C. Quantification of GATA3-mCherry reporter area. Note that Y-axis scale differs between B and C, as GATA3 area represents a substantially smaller proportion of the organoid in most cases. D. GATA3 area as a proportion of total measured reporter area (MAFB+GATA3), highlighting a shift in R0 toward a more distalised fate. E. The total number of individual organoids used for quantification, by set and condition.

FIG. 10. Analysis of single cell RNA datasets. A. Variability within the datasets represented as a UMAP plot, coloured by transcriptional cluster, predicted cell cycle phase, main cell type and organoid conformation (clockwise from top left). B. Marker genes of main cells type, WT1 and PAX2 (nephron), PDGFRA (stroma) and SOX17 (endothelial). C. Proportion of each cell type in replicate conditions. P value (one-way ANOVA) indicated if p<0.2. D. UMAP representation of nephron cells after re-transformation and clustering at higher resolution. Plots are coloured by transcriptional cluster, predicted cell cycle phase and organoid conformation. Cluster identities are stated. E. Marker genes identifying each cluster: GATA3 (distal), HNF1B (pre-tubule), CUBN (proximal), HNF4A (proximal), FOXC2 (pre-podocyte), MAFB (pre-podocyte/podocyte), PODXL (podocyte), SIX2 (progenitor), EYA1 (progenitor). F. Stromal UMAP coloured by transcriptional cluster, predicted cell cycle phase and organoid conformation (top to bottom). G. Markers of specific stromal clusters; SIX2, LYPD1, FOXC2, HOXA11 (Cluster 3, nephron progenitor-like), WNT5A, LHX9 (Cluster 7) and ZIC1 and ZIC4 (Cluster 10). H. Heatmap of scaled log counts per million of pseudo bulk counts from scRNAseq sets for the top 100 most significantly expressed genes identified in bulk RNAseq analysis (FIG. 7). Each column represents a single cluster from a single replicate (e.g. R40, Nephron, Set1). Hierarchical clustering of the limited gene set indicates that bulk-RNAseq changes are largely driven by changes in the nephrons and endothelial cells.

FIG. 11. Generation of a kidney tissue patch using 3D extrusion cellular bio-printing. A. Illustration of the scripted movement of the needle tip for cell paste extrusion, generating a patch organoid across an area of approximately 4.8 mm×6 mm, containing approximately 4×10⁵ cells. Lines indicate continuous movements. B. Brightfield imaging of the bio-printed kidney tissue patch demonstrating uniform formation of nephron structures, including at the edge and within the centre of the patch. C. Live confocal imaging of MAFB^{mTAGBFP2} reporter signal throughout a patch organoid at D7+12 of culture. Scale bar represents 1 mm. D. Confocal immunofluorescence of a D7+14 patch organoid demonstrating uniform distribution of nephrons expressing markers for podocytes of the glomeruli (mTagBFP2 [left panel; blue), proximal tubules (LTL [left panel; green] and HNF4A [right panel; red]), nephron epithelium (EPCAM [left panel; red]), distal tubule/loop of Henle TAL (SLC12A1 [right panel; green]) and endothelial cells (SOX17 [right panel; grey]). Scale bars represent 100 μm. E. Live confocal imaging of a D7+14 patch organoid derived from the HNF4A^{YFP} reporter iPSC line following incubation in TRITC-albumin substrate. Images depict TRITC-albumin (red) uptake into YFP-positive proximal tubules (yellow). Outlined areas in top panels (whole organoid images) are shown at higher magnification in lower panels, with and without phase contrast overlays. Scale bars represent 100 μm.

FIG. 12. MAFB^{mTagBFP2} reporter expression in organoids correlates to total nephron number. A,B) Examples of low resolution, high throughput imaging used to quantify MAFB area as a proxy for nephron volume in organoids. Brightfield and MAFB^{mTagBFP2} signal was captured for each organoid using a low NA 4× objective with a spinning disk system, enabling fast capture of many samples. With a large axial depth of field, these images capture the majority of signal within each organoid in a single plane. Given the similarity in thickness (E,F, FIG. 5) this planar area is approximately proportional to total MAFB+ glomerular volume and hence correlates to nephron number. A portion of an example image used for quantification of R0 (A) and R40 (B) organoids at D7+12 is shown. Note R40 organoids are much longer and were captured by stitching multiple image fields. Only a small portion of the organoid is shown. C, D) Samples were fixed and stained at D7+12 for MAFB^{mTagBFP2} reporter (Cyan), mature podocyte marker NPHS1 (Red) and atypical protein kinase C (aPKC, Green), a marker of the apical cell membrane. Each nephron consists of a rounded glomerular structure containing podocytes (examples highlighted by white arrows) connected to other tubular segments that are marked by aPKC but lack NPHS1. Nephrons are seen throughout the field and are packed together so that individual nephrons cannot be easily separated visually. MAFB^{mTagBFP2} reporter is expressed specifically in NPHS1 expressing podocytes but is absent from other nephron segments (aPKC⁺, NPHS1⁻ regions) or from other cell types. Images are maximum projections (50 μm span). E,F) Both conditions have a similar axial morphology in nephron-containing regions when viewed as an orthogonal slice (i.e. along the imaging Z-axis). A single orthogonal slice rendered from a 3D stack is shown. G,H) Cropped high-resolution fields showing a single glomerulus for each condition confirm co-expression of MAFB^{mtagBFP2} reporter and NPHS1 in podocytes. A single confocal slice is shown. All images are representative of at least n=3 stained samples.

FIG. 13. The spatial distribution of stromal markers by wholemount immunofluorescence. A-C) Immunofluorescence staining for markers of organoid stromal populations based on scRNA profiling. R0 organoids consist of a nephron containing area (Nephrons), a central role (Core) where nephrons are largely absent, and a thin edge (Thin edge) of monolayer cells that are typically not observed in brightfield imaging. R40 line organoids are primarily composed of a dense nephron-containing region and a thin monolayer edge, with no central core. Stromal population markers (A) MEIS1/2/3, (B) SIX1 and (C) SOX9 are present in the areas surrounding nephrons, and within the thin monolayer sheet at the edge of each organoid, but are largely absent from the central core of R0 organoids. Representative images from n=3 organoids stained per condition are shown. Images are maximum projections spanning the full volume of the organoid. D) UMAP plots representing stromal cells in scRNA datasets, colour coded to show expression of MEIS1, MEIS2, SIX1 and SOX9. These combined markers include most of the cells in the dataset, suggesting that the absence of staining in the central core observed in (E) may indicate low overall cellularity in that region.

FIG. 14. Direct comparison between kidney organoids and human fetal kidney confirms improved maturation of proximal tubules within R40 bio-printed lines. A) UMAP plots comparing transcriptional identity based on unbiased clustering in Seurat (left) and prediction using the scPred method to classify cells according to their similarity to a human fetal kidney (HFK) dataset (right). Identity is assigned based on the most similar cell type in the human fetal kidney data. B) The proportion of cells assigned to each cell type identity across replicates. Points show individual replicate values colour coded by replicate barcode (where HTO-1 is Set 1). Bars show SEM. P-values based on one-way ANOVA indicate a significant difference in the number of cells predicted to be Pre-Pod cells, with greatest abundance in the R40 datasets. Bio-printed conditions (R40 and R0) have more cells predicted to be podocytes, and less distal and pre-tubule cells. However, these changes were not significant. These results support the trends observed in the analysis presented in FIG. 5. C) The distribution of maximum similarity scores for the classification of each cell across conformations, plotted by cell type predicted. Most cells show a high similarity to the predicted fetal kidney cell type. D) Genes identified as significantly increased in R40 versus Manual organoids (SLC51B, FABP3 and SULT1E1) are expressed in the mature proximal tubule cells of human fetal kidney, confirming that these genes are associated with a more mature cell type. A gene that was significantly decreased in R40 vs Manual organoids (SPP1) is expressed selectively in less mature cell types, further confirming increased maturity in R40 proximal cells. UMAP shows transcriptional identity in human fetal kidney data. Top left plot is colour coded by human fetal kidney cell types specific to developing (renal vesicle and comma shaped body [RV_CSB], blue; proximal early nephron [PEN], red) and mature proximal tubule (PT, green). Lower left plot shows a ‘dot plot’ style representation of selected gene where size indicates the percentage of HFK cells expressing the gene and colour indicates normalised expression level. Normalised expression of each gene per cell is indicated on individual UMAP plots where expression is colour coded.

### DESCRIPTION OF EMBODIMENTS

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described.

As used herein, except where the context requires otherwise, the term “comprise” and variations of the term, such as “comprising”, “comprises” and “comprised”, are not intended to exclude further additives, components, integers or steps.

It will be appreciated that the indefinite articles “a” and “an” are not to be read as singular indefinite articles or as otherwise excluding more than one or more than a single subject to which the indefinite article refers. For example, “a” cell includes one cell, one or more cells and a plurality of cells.

As used herein, “bio-ink” means a liquid, semi-solid, or solid composition for use in bio-printing. In some embodiments, bio-ink comprises cell solutions, cell aggregates, cell comprising gels, or multicellular bodies. In some embodiments, the bio-ink additionally comprises support material. In some embodiments, the bio-ink additionally comprises non-cellular materials that provide specific biomechanical properties that enable bio-printing. In some embodiments the bio-ink comprises an extrusion compound. In some embodiments, the bio-ink additionally comprises an additive to increase the viscosity of the bio-ink and reduce cell settling prior to bio-printing. Examples of suitable additives include hydrogel and hyaluronic acid.

As used herein, “bio-printing,” “bio-printed,” “bio-printing,” or “bio-printed” means utilizing three-dimensional, precise deposition of cells (e.g., cell solutions, cell-containing gels, cell suspensions, cell concentrations, multicellular aggregates, multicellular bodies, etc.) via methodology that is compatible with an automated or semi-automated, computer-aided, three-dimensional prototyping device (e.g., a bio-printer). In this instance, this does not refer to robotic liquid handling but to extrusion or additive bio-printing. Any suitable bio-printer, capable of extrusion bio-printing for the precise deposition of a bio-ink comprising cells may be utilized for bio-printing of this invention. The bio-printer may, for example, be an extrusion bio-printer where the cells are extruded as cells only or as cells suspended within a material, which may include a hydrogel, biological matrix or other chemical compound compatible with cell viability. An example of a suitable bio-printer includes the Novogen Bio-Printer® from Organovo, Inc. (San Diego, Calif.). As used herein, bio-printed kidney tissue refers to a kidney organoid which has been prepared through the process of bio-printing and the terms “bio-printed kidney tissue” and “bio-printed kidney organoid” may be used interchangeably.

The terms “differentiate”, “differentiating” and “differentiated”, relate to progression of a cell from an earlier or initial stage of a developmental pathway to a later or more mature stage of the developmental pathway. It will be appreciated that in this context “differentiated” does not mean or imply that the cell is fully differentiated and has lost pluripotency or capacity to further progress along the developmental pathway or along other developmental pathways. Differentiation may be accompanied by cell division.

As used herein, the term “extrusion bio-printing” refers to utilizing three-dimensional, precise extrusion of cells (e.g., cell solutions, cell-containing gels, cell suspensions, cell concentrations, multicellular aggregates, multicellular bodies, etc.) with an automated or semi-automated, computer-aided, three-dimensional prototyping device (e.g., a bio-printer). Extrusion bio-printing provides control over cell aggregate shape, cell number, cell density and final tissue height (thickness) by introducing fine tip movement as cells are extruded. Via scripting of the movement of the extrusion port during the process of extrusion, the bio-ink can be spread over a defined distance and in a specific configuration in a way that would not be possible to control, or at least reproduce with accuracy, manually. Increasing the amount of tip movement for a given rate of cell extrusion (ratio) enables the user to create bio-printed tissue of variable cell density, shape and thickness as cells are spread, and subsequently aggregate, over larger surface areas.

As used herein, the terms “induce”, “inducing”, “induced” and “induction” in reference to a cell or plurality of cells, or bio-ink (including printed bio-ink), relate to promoting the differentiation, development or maturation of the cell or plurality of cells or bio-ink (including printed bio-ink). For example, inducing can involve treating or culturing a cell or plurality of cells or bio-ink (including printed bio-ink) for a time and under conditions to permit a change from a default genotype and/or phenotype to a different or non-default genotype and/or phenotype. In the context of promoting the differentiation, development or maturation of a cell or plurality of cells or bio-ink (including printed bio-ink) to form bio-printed kidney tissue this includes causing a cell or a plurality of cells to express one or more markers associated with kidney tissue, or to divide into progeny cells expressing one or more markers associated with kidney tissue, that are different from the original identity of the cell or cells, such as genotype (i.e. change in gene expression as determined by genetic analysis such as a PCR or microarray) and/or phenotype (i.e. change in morphology, function and/or expression of a protein). In one example, “inducing” includes promoting the differentiation, development or maturation of one or more nephron progenitor cells to nephron epithelia such as one or more of connecting segment, distal convoluted tubule (DCT) cells, distal straight tubule (DST) cells, proximal convoluted (PCT) and straight tubules (PST) segments 1, 2 and 3, PCT and PST cells, podocytes, glomerular endothelial cells, ascending Loop of Henle and/or descending Loop of Henle. In one example, “inducing” includes causing an increase in expression of one or more of SLC12A1, CDH1, HNF4A, CUBN, LRP2, EPCAM and MAFB. The step of inducing may include contacting the bio-printed bio-ink with particular growth factors (for example FGF-9) for a period of time sufficient to form kidney tissue. In some examples, the step of inducing may also include contacting the bio-ink with particular growth factors (for example CHIR) for a sufficient period of time before the bio-ink is bio-printed and further cultured.

As used herein, except where the context requires otherwise, the term “height” means the tissue height or micromass height. In one example, the term “height” as used in terms of the “the height of the bio-printed kidney tissue” means the height of the tissue from the surface upon which the tissue is deposited, and refers to the final tissue height. In another example, the term “height” is used in terms of “the height of a layer of bio-printed bio-ink” and means the height of the cell mass or the micromass in the layer. In yet another example, the term “high” is used to specify that “the bio-ink is bio-printed in a layer that is about X μm high” and means the cell mass or micromass in the layer is X μm high. Where the bio-ink additionally comprises additives, additional compounds or materials (such as support material, non-cellular materials, an extrusion compound or an additive), the height of the layer of bio-printed bio-ink refers to the height of the cell mass or micromass and not the height of the bio-ink itself. The height that is measured is the height of the layer of settled cell mass or micromass from the surface upon which the tissue is deposited.

A “progenitor cell” is a cell which is capable of differentiating along one or a plurality of developmental pathways, with or without self-renewal. Typically, progenitor cells are unipotent or oligopotent and are capable of at least limited self-renewal.

As will be well understood in the art, the stage or state of differentiation of a cell may be characterized by the expression and/or non-expression of one of a plurality of markers. In this context, by “markers” is meant nucleic acids or proteins that are encoded by the genome of a cell, cell population, lineage, compartment or subset, whose expression or pattern of expression changes throughout development. Nucleic acid marker expression may be detected or measured by any technique known in the art including nucleic acid sequence amplification (e.g. polymerase chain reaction) and nucleic acid hybridization (e.g. microarrays, Northern hybridization, in situ hybridization), although without limitation thereto. Protein marker expression may be detected or measured by any technique known in the art including flow cytometry, immunohistochemistry, immunoblotting, protein arrays, protein profiling (e.g. 2D gel electrophoresis), although without limitation thereto.

As used herein “nephron progenitor cells” are progenitor cells derived from metanephric mesenchyme that can differentiate into all nephron segments (other than collecting duct) via an initial mesenchyme to epithelial transition, which include nephron epithelia such as connecting segment, distal convoluted tubule (DCT) cells, distal straight tubule (DST) cells, proximal convoluted and straight tubule segments 1, 2 and 3 (PCT/PST), PCT and PST cells, podocytes, glomerular endothelial cells, ascending Loop of Henle and/or descending Loop of Henle, although without limitation thereto. Nephron progenitor cells are also capable of self-renewal.

Non-limiting examples of markers characteristic or representative of metanephric mesenchyme (MM) include WT1, SALL1, GDNF and/or HOXD11, although without limitation thereto. Non-limiting examples of markers characteristic or representative of nephron progenitor cells include WT1, SIX1, SIX2, CITED1, PAX2, GDNF, SALL1, OSR1 and HOXD11, although without limitation thereto.

By “ureteric epithelial progenitor cell” is meant an epithelial progenitor cell derived, obtainable or originating from mesonephric duct or its derivative ureteric bud that can develop into kidney tissues and/or structures such as the collecting duct.

Non-limiting examples of characteristic or representative markers of ureteric epithelial progenitor cells include WNT9B, RET, GATA3, CALB1, E-CADHERIN and PAX2, although without limitation thereto.

As hereinbefore described, the nephron progenitor cells and ureteric epithelial progenitor cells are differentiated from intermediate mesoderm (IM) cells in the presence of FGF9 alone or in combination with one or more agents that include BMP7, retinoic acid (RA), agonist or analog, an RA antagonist such as AGN193109 and/or FGF20 and preferably heparin.

By “intermediate mesoderm (IM)” cells is meant embryonic mesodermal cells that arise from definitive mesoderm which in turn is derived from posterior primitive streak and can ultimately develop into the urogenital system, inclusive of the ureter and kidney and other tissues such as gonad. Non-limiting examples of markers characteristic or representative of intermediate mesoderm include PAX2, OSR1 and/or LHX1.

It will also be appreciated that production of IM cells is not meant to imply that the TM cells are a pure or homogeneous population of IM cells without other cell types being present (such as definitive mesoderm). Accordingly, reference to “TM cells” or a “population of IM cells” means that the cells or cell population comprise(s) TM cells. Suitably, according to the invention IM cells are produced by contacting posterior primitive streak cells with one or more agents that facilitate differentiation of the posterior primitive streak cells into IM cells, as will be described in more detail hereinafter.

Preferably, the TM cells are produced by contacting posterior primitive streak cells with one or more agents that facilitate differentiation of the posterior primitive streak cells into IM cells.

By “posterior primitive streak (PPS)” cells is meant cells obtainable from, or cells functionally and/or phenotypically corresponding to, cells of the posterior end of a primitive streak structure that forms in the blastula during the early stages of mammalian embryonic development. The posterior primitive streak establishes bilateral symmetry, determines the site of gastrulation and initiates germ layer formation. Typically, posterior primitive streak is the progenitor of mesoderm (i.e. presumptive mesoderm) and anterior primitive streak is the progenitor of endoderm (i.e. presumptive endoderm). Non-limiting examples of markers characteristic or representative of posterior primitive streak include Brachyury (T). A non-limiting example of a marker characteristic or representative of anterior primitive streak is SOX17. MIXL1 may be expressed by both posterior and anterior primitive streak.

It will also be appreciated that production of posterior primitive streak cells is not meant to imply that the posterior primitive streak cells are a pure or homogeneous population of posterior primitive streak cells without other cell types being present. Accordingly, reference to “posterior primitive streak cells” or a “population of posterior primitive streak cells” means that the cells or cell population comprise(s) posterior primitive streak cells.

The terms “human pluripotent stem cell” and “hPSC” refer to cells derived, obtainable or originating from human tissue that display pluripotency. The hPSC may be a human embryonic stem cell or a human induced pluripotent stem cell.

Human pluripotent stem cells may be derived from inner cell mass or reprogrammed using Yamanaka factors from many fetal or adult somatic cell types. The generation of hPSCs may be possible using somatic cell nuclear transfer.

The terms “human embryonic stem cell”, “hES cell” and “hESC” refer to cells derived, obtainable or originating from human embryos or blastocysts, which are self-renewing and pluri- or toti-potent, having the ability to yield all of the cell types present in a mature animal. Human embryonic stem cells (hESCs) can be isolated, for example, from human blastocysts obtained from human in vivo preimplantation embryos, in vitro fertilized embryos, or one-cell human embryos expanded to the blastocyst stage.

The terms “induced pluripotent stem cell” and “iPSC refer to cells derivable, obtainable or originating from human adult somatic cells of any type reprogrammed to a pluripotent state through the expression of exogenous genes, such as transcription factors, including a preferred combination of OCT4, SOX2, KLF4 and c-MYC. hiPSC show levels of pluripotency equivalent to hESC but can be derived from a patient for autologous therapy with or without concurrent gene correction prior to differentiation and cell delivery.

More generally, the method disclosed herein could be applied to any pluripotent stem cell derived from any patient or a hPSC subsequently modified to generate a mutant model using gene-editing or a mutant hPSC corrected using gene-editing. Gene-editing could be by way of CRISPR, TALEN or ZF nuclease technologies.

As used herein, “tissue” means an aggregate of cells. In some embodiments, the cells in the tissue are cohered or fused.

As used herein, “scaffold” refers to synthetic scaffolds such as polymer scaffolds and porous hydrogels, non-synthetic scaffolds such as pre-formed extracellular matrix layers, dead cell layers, and decellularized tissues, and any other type of pre-formed scaffold that is integral to the physical structure of the engineered tissue and not able to be removed from the tissue without damage/destruction of said tissue. In further embodiments, decellularized tissue scaffolds include decellularized native tissues or decellularized cellular material generated by cultured cells in any manner; for example, cell layers that are allowed to die or are decellularized, leaving behind the extracellular matrix (ECM) they produced while living.

As used herein an “individual” is an organism of any mammalian species including but not limited to humans, primates, apes, monkey, dogs, cats, mice, rats, rabbits, pigs, horses and others. A subject can be any mammalian species alive or dead.

As used herein, “about” or “approximately” means±10% of the recited value. For example, about 10 includes 9-11.

The term “and/or”, e.g., “X and/or Y” shall be understood to mean either “X and Y” or “X or Y” and shall be taken to provide explicit support for both meanings or for either meaning.

### Bio-Printed Kidney Tissue

Herein disclosed is bio-printed kidney tissue comprising a bio-ink, wherein the bio-ink comprises a plurality of cells, and wherein the bio-ink is bio-printed in a layer that is about 150 high or less and wherein the bio-printed bio-ink is induced to form kidney tissue. In one embodiment, the bio-ink is bio-printed in a layer selected from about 15 μm to about 150 In one embodiment, the bio-ink is bio-printed in a layer selected from about 25 μm high to about 100 μm high. In a preferred embodiment the bio-ink is bio-printed in a layer about 50 μm high or less. In one embodiment, the bio-ink is bio-printed in a layer about 15 μm high. In one embodiment, the bio-ink is bio-printed in a layer about 20 μm high. In one embodiment, the bio-ink is bio-printed in a layer about 25 μm high. In one embodiment, the bio-ink is bio-printed in a layer about 30 μm high. In one embodiment, the bio-ink is bio-printed in a layer about 35 high. In one embodiment, the bio-ink is bio-printed in a layer about 40 μm high. In one embodiment, the bio-ink is bio-printed in a layer about 50 μm high. In one embodiment, the bio-ink is bio-printed in a layer about 60 μm high. In one embodiment, the bio-ink is bio-printed in a layer about 70 μm high. In one embodiment, the bio-ink is bio-printed in a layer about 80 high. In one embodiment, the bio-ink is bio-printed in a layer about 90 μm high. In one embodiment, the bio-ink is bio-printed in a layer about 100 μm high.

In one embodiment, the height of the bio-printed kidney tissue is about 150 μm or less. In other words, the height of the final bio-printed kidney tissue after the bio-printed bio-ink is induced to form kidney tissue is about 150 μm or less. In another embodiment, the height of the bio-printed kidney tissue is from about 50 μm to about 150 In another embodiment, the height of the bio-printed kidney tissue is from about 100 μm to about 150 μm.

In one embodiment, the bio-printed layer of bio-ink comprises between about 5,000 and about 100,000 cells per mm². In one embodiment, the bio-printed layer of bio-ink comprises between about 10,000 and about 50,000 cells per mm². In one embodiment, the bio-printed layer of bio-ink comprises between about 5,000 and about 20,000 cells per mm². In one embodiment, the bio-printed layer of bio-ink comprises between about 10,000 and about 15,000 cells per mm². In one embodiment, the bio-printed layer of bio-ink comprises about 5,000 cells per mm². In one embodiment, the bio-printed layer of bio-ink comprises about 10,000 cells per mm². In one embodiment, the bio-printed layer of bio-ink comprises about 15,000 cells per mm². In one embodiment, the bio-printed layer of bio-ink comprises about 20,000 cells per mm². In one embodiment, the bio-printed layer of bio-ink comprises about 30,000 cells per mm². In one embodiment, the bio-printed layer of bio-ink comprises about 40,000 cells per mm². In one embodiment, the bio-printed layer of bio-ink comprises about 50,000 cells per mm². In one embodiment, the bio-printed layer of bio-ink comprises about 60,000 cells per mm². In one embodiment, the bio-printed layer of bio-ink comprises about 70,000 cells per mm². In one embodiment, the bio-printed layer of bio-ink comprises about 80,000 cells per mm². In one embodiment, the bio-printed layer of bio-ink comprises about 90,000 cells per mm². In one embodiment, the bio-printed layer of bio-ink comprises about 100,000 cells per mm².

According to a preferred embodiment, the bio-printed kidney tissue comprises a bio-printed layer of bio-ink comprising from about 10,000 cells to about 20,000 cells per mm² and having a height of about 50 μm or less when printed. In a further preferred embodiment, the bio-printed kidney tissue comprises a bio-printed layer of bio-ink comprising about 20,000 cells per mm² and having a height of about 40 μm or less when printed. In a further preferred embodiment, the bio-printed kidney tissue comprises a bio-printed layer of bio-ink comprising about 14,000 cells per mm² and having a height of about 30 μm or less, when printed. In a further preferred embodiment, the bio-printed kidney tissue comprises a bio-printed layer of bio-ink comprising about 11,000 cells per mm² and having a height of about 25 μm or less when printed. In a further preferred embodiment, the bio-printed kidney tissue comprises a bio-printed layer of bio-ink comprising about 10,000 cells per mm² and having a height of about 20 μm or less when printed.

In one embodiment, the bio-ink comprises between approximately 10,000 cells/μl and approximately 400,000 cells/μl. In one embodiment, the bio-ink comprises between about 10,000 cells/μl and about 100,000 cells/μl. In one embodiment, the bio-ink comprises between about 100,000 cells/μl and about 400,000 cells/μl. In one embodiment, the bio-ink comprises between about 50,000 cells/μl and about 200,000 cells/μl. In one embodiment, the bio-ink comprises about 10,000 cells/μl, about 30,000 cells/μl, about 40,000 cells/μl, about 50,000 cells/μl, about 60,000 cells/μl, about 70,000 cells/μl, about 80,000 cells/μl, about 90,000 cells/μl, about 100,000 cells/μl, about 150,000 cells/μl, about 200,000 cells/μl, about 250,000 cells/μl, about 300,000 cells/μl, or about 400,000 cells/μl. In a preferred embodiment, the bio-ink comprises about 200,000 cells/μl.

In some embodiments, the bio-ink comprises partly differentiated cells. In some embodiments, the bio-ink comprises fully differentiated cells.

In some embodiments, the bio-ink comprises cells differentiated from human stem cells (HSCs), including but not limited to, human induced pluripotent stem cells (iPSCs) and human embryonic stem cells (hESCs). In some embodiments, the bio-ink comprises primitive streak cells, including but not limited to posterior primitive streak cells. In some embodiments, the bio-ink comprises intermediate mesoderm (IM) cells. In some embodiments, the bio-ink comprises metanephric mesenchyme (MM) cells. In some embodiments, the bio-ink comprises nephric duct cells. In some embodiments, the bio-ink comprises renal progenitor cells, including but not limited to nephron progenitor cells, ureteric epithelial progenitor cells, or a combination thereof.

In some embodiments, the cells of the bio-ink comprise patient-derived cells. In some embodiments, the cells of the bio-ink comprise gene-edited cells. In some embodiments, the cells of the bio-ink comprise patient-derived cells that are also gene-edited cells. In some embodiments, the cells of the bio-ink comprise cells from a reporter line. In some embodiments, the cells of the bio-ink comprise a reporter line cell that is also gene edited.

In some embodiments, the cells of the bio-ink comprise normal healthy cells. In some embodiments, the cells of the bio-ink comprise kidney disease patient cells. In some embodiments, the cells of the bio-ink comprise a combination of patient cells and healthy cells.

In one example, the bio-printed kidney tissue is derived from a culture expanded population of renal progenitor cells, such as nephron progenitor cells.

In another example, the bio-printed kidney tissue is derived from a culture expanded population of MM cells or IM cells that are characterized by the method used for culture expansion and/or production.

In an example, the renal progenitor cells are produced by contacting posterior primitive streak cells with one or more agents that facilitate differentiation of the posterior primitive streak cells into renal progenitor cells, such as IM cells or MM cells. In an example, the method of producing renal progenitor cells comprises, culturing a population of stem cells for around 2 to 5 days in a cell culture medium comprising a Wnt/β-catenin agonist followed culturing the cells for around 2 to 5 days in a cell culture medium comprising FGF such as FGF9. In an example, the method of producing renal progenitor cells comprises, culturing a population of stem cells for around 2 to 5 days in a cell culture medium comprising a Wnt/β-catenin agonist followed culturing the cells for around 3 to 4 days in a cell culture medium comprising FGF such as FGF9. In this example, the cells may be cultured for 7 days or more, after which the renal progenitor cells are dissociated. In this example, the renal progenitor cells may be printed around day 10 to 13. In another example, the method of producing renal progenitor cells comprises, culturing a population of stem cells for around 2 to 5 days in a cell culture medium comprising a Wnt/β-catenin agonist followed by culturing the cells for around 3 to 4 days in a cell culture medium comprising FGF such as FGF9. In another example, the renal progenitor cells may be cultured in a nephron progenitor maintenance media until around day 10 to 14 before the renal progenitor cells are printed.

In another example, the bio-printed kidney tissue is derived from a culture expanded population of IM cells that are characterized by the method used for culture expansion and/or production and bio-printed according to the methods described herein.

Accordingly, in an example, the method of producing IM cells comprises, culturing a population of stem cells for around 3 to 5 days in a cell culture medium comprising a Wnt/β-catenin agonist followed culturing the cells for around 2 to 5 days in a cell culture medium comprising FGF such as FGF9. In an example, the method of producing IM cells comprises, culturing a population of stem cells for around 3 to 5 days in a cell culture medium comprising a Wnt/β-catenin agonist followed culturing the cells for around 3 to 5 days in a cell culture medium comprising FGF such as FGF9. In these examples, the cells can be cultured 7 days in total, after which the IM cells are dissociated. The term “Wnt/β-catenin agonist” is used in the context of the present disclosure to refer to a molecule that inhibits GSK3 (e.g. GSK3-β) in the context of the canonical Wnt signalling pathway, but preferably not in the context of other non-canonical, Wnt signalling pathways. Examples of Wnt β-catenin agonists include recombinant WNT3A, CHIR99021 (CHIR), LiCl SB-216763, CAS 853220-52-7 and other Wnt/β-catenin agonists that are commercially available from sources such as Santa Cruz Biotechnology and R & D Systems.

In an example, the IM cells are produced by culturing stem cells for 7 days, wherein days 3 to 5 involve culturing stem cells in cell culture medium comprising an above referenced high concentration of CHIR and the remaining days involve culturing cells in cell culture medium comprising an above referenced concentration of an FGF. For example, the IM cells can be produced by culturing stem cells for 7 days, wherein days 3 to 5 involve culturing stem cells in cell culture medium comprising at least 3 μM CHIR and the remaining days involve culturing cells in cell culture medium comprising at least 100 ng/ml FGF9.

In another example, IM cells can be produced by culturing stem cells for up to 13 days, after which the IM cells are dissociated. In another example, IM cells can be produced by culturing stem cells for 8 days. In another example, IM cells can be produced by culturing stem cells for 9 days. In another example, IM cells can be produced by culturing stem cells for 10 days. In another example, IM cells can be produced by culturing stem cells for 11 days. In another example, IM cells can be produced by culturing stem cells for 12 days. In another example, IM cells can be produced by culturing stem cells for 13 days. In another example, IM cells can be produced by culturing stem cells for 14 days. In another example, IM cells can be produced by culturing stem cells for 15 days. In another example, IM cells can be produced by culturing stem cells for more than 10 days. In each of these examples, days 3 to 5 can involve culturing stem cells in cell culture medium comprising at least 3 μM CHIR, wherein cells are cultured in cell culture medium comprising FGF9 for the remaining days. For example, days 3 to 5 can involve culturing stem cells in cell culture medium comprising between 3 μM and 8 μM CHIR, wherein cells are cultured in cell culture medium comprising FGF9 for the remaining days.

In an example, cells are cultured in cell culture media comprising between 3 and 8 μM of a Wnt/β-catenin agonist before they are cultured in cell culture media comprising FGF. In another example, cells are cultured in cell culture media comprising 4 μM of a Wnt/β-catenin agonist before they are cultured in cell culture media comprising FGF. In another example, cells are cultured in cell culture media comprising 5 μM of a Wnt/β-catenin agonist before they are cultured in cell culture media comprising FGF. In another example, cells are cultured in cell culture media comprising 6 μM of a Wnt/β-catenin agonist before they are cultured in cell culture media comprising FGF. In another example, cells are cultured in cell culture media comprising 7 μM of a Wnt/β-catenin agonist before they are cultured in cell culture media comprising FGF. In another example, cells are cultured in cell culture media comprising 8 μM of a Wnt/β-catenin agonist before they are cultured in cell culture media comprising FGF. In these examples the Wnt/β-catenin agonist can be CHIR. For example, cells can be cultured in cell culture media comprising 3 to 8 μM of CHIR before they are cultured in cell culture media comprising FGF.

In an example, the IM cell culture medium comprises at least 50 ng/ml FGF9. In another example, the cell culture medium comprises at least 100 ng/ml FGF9. In another example, the cell culture medium comprises at least 150 ng/ml FGF9. In another example, the cell culture medium comprises at least 200 ng/ml FGF9. In another example, the cell culture medium comprises at least 300 ng/ml FGF9. In another example, the cell culture medium comprises at least 350 ng/ml FGF9. In another example, the cell culture medium comprises at least 400 ng/ml FGF9. In another example, the cell culture medium comprises at least 500 ng/ml FGF9. In another example, the cell culture medium comprises between 50 ng and 400 ng/ml FGF9. In another example, the cell culture medium comprises between 50 ng and 300 ng/ml FGF9. In another example, the cell culture medium comprises between 50 ng and 250 ng/ml FGF9. In another example, the cell culture medium comprises between 100 ng and 200 ng/ml FGF9.

In another example, an above referenced level of FGF9 is substituted for FGF2. For example, the IM cell culture medium can comprise between 50 ng and 400 ng/ml FGF2. In another example, the cell culture medium comprises between 50 ng and 300 ng/ml FGF2. In another example, the cell culture medium comprises between 50 ng and 250 ng/ml FGF2. In another example, the cell culture medium comprises between 100 ng/ml and 200 ng/ml FGF2.

In another example, an above referenced level of FGF9 is substituted for FGF20. For example, the IM cell culture medium can comprise between 50 ng and 400 ng/ml FGF20. In another example, the cell culture medium comprises between 50 ng and 300 ng/ml FGF20. In another example, the cell culture medium comprises between 50 ng and 250 ng/ml FGF20. In another example, the cell culture medium comprises between 100 ng/ml and 200 ng/ml FGF20.

In an example, the IM cell culture medium which comprises FGF also comprises heparin. In an example, the cell culture medium comprises 0.5 μg/ml heparin. In another example, the cell culture medium comprises 1 μg/ml heparin. In another example, the cell culture medium comprises 1.5 μg/ml heparin. In another example, the cell culture medium comprises 2 μg/ml heparin. In another example, the cell culture medium comprises between 0.5 μg/ml and 2 μg/ml heparin. In another example, the cell culture medium comprises between 0.5 μg and 1.5 μg/ml heparin. In another example, the cell culture medium comprises between 0.8 μg/ml and 1.2 μg/ml heparin.

In an example, the bio-ink is induced to form kidney tissue by contacting the bio-ink with FGF-9. In another example, the bio-ink is induced to form kidney tissue by contacting the bio-ink with FGF-9 for a period of 5 days. In some examples, the plurality of cells may be briefly contacted with a cell culture medium comprising CHIR before being bio-printed and further cultured. For example, the plurality of cells can be contacted with a cell culture medium comprising 3 to 8 μM CHIR for one to two hours before being bio-printed and further cultured. In another example, plurality of cells can be contacted with a cell culture medium comprising 5 μM CHIR for one hour before being bio-printed and further cultured.

In other examples, IM or MM cells used to produce bio-printed kidney tissue can be cultured in culture mediums comprising different or additional components. Exemplary components and timing for their use in cell culture is discussed below.

In an example, the cell culture medium can comprise a Rho kinase inhibitor (ROCKi) such as Y-27632 (StemCell Technologies). In this example, stem cells are cultured in a cell culture medium comprising ROCKi for 24 hours before being cultured in a cell culture medium comprising at least 4 μM CHIR for around 3 to 4 days. In this example, cells can subsequently be cultured in a cell culture medium comprising FGF for a further 3 to 4 days. In an example, the cell culture medium can comprise 8 μM ROCKi. In another example, the cell culture medium can comprise 10 μM ROCKi. In another example, the cell culture medium can comprise 12 μM ROCKi. In another example, the cell culture medium can comprise between 8 μM and 12 μM ROCKi.

In an above example, after culturing with ROCKi for 24 hours and at least 4 μM CHIR for around 3 to 4 days, the cells can be cultured in a culture medium which comprises FGF9 and one or more or all of a Wnt/β-catenin agonist such as CHIR at a low concentration (e.g. less than 3 μM), an above referenced concentration of Heparin, poly(vinyl alcohol) (PVA) and methyl cellulose (MC). In this example, the IM cell culture medium can comprise at least 0.05% PVA. In another example, the cell culture medium comprises 0.1% PVA. In another example, the cell culture medium comprises 0.15% PVA. In another example, the cell culture medium comprises between 0.1% and 0.15% PVA. In an example, the cell culture medium can comprise at least 0.05% MC. In another example, the cell culture medium comprises 0.1% MC. In another example, the cell culture medium comprises 0.15% MC. In another example, the cell culture medium comprises between 0.1% and 0.15% MC.

In an example, the bio-printed kidney tissue is derived by producing IM cells using an above referenced method, dissociating the IM cells, preparing a bio-ink, bio-printing the bio-ink and then further culturing the bio-ink, i.e. the bio-printed cells in a method of producing a bio-printed kidney tissue discussed hereinbelow. For example, IM cells can be produced using an above exemplified method, dissociated and then bio-printed to form kidney tissue. In examples, bio-printing can be performed in culture on a supported filter. For example, IM cells can be produced using an above exemplified method, dissociated and then cultured for a subsequent period post bio-printing (e.g. 12 days) on Transwell™ filters.

In an example, the plurality of cells can be dissociated using EDTA after culturing under conditions and for a duration sufficient to produce the target renal cell progenitors. In an example, IM cells can be dissociated using EDTA. In another example, cells can be dissociated using trypsin or TrypLE or Accutase or Collagenase. In an example, cells are cultured for at least 12 days after bio-printing. In another example, cells are cultured for at least 13 days after bio-printing. In another example, cells are cultured for at least 14 days after bio-printing. In another example, cells are cultured for at least 15 days after bio-printing. In another example, cells are cultured for at least 20 days after bio-printing. In another example, cells are cultured for at least 25 days after bio-printing. In another example, cells are cultured for at least 35 days after bio-printing.

In an example, the plurality of cells is dissociated after a duration in culture sufficient to produce the target renal cell progenitors. In this example, the dissociated cells are then bio-printed to produce bio-printed kidney tissue. In an example, IM cells are dissociated after 7 days in culture (d7) and then bio-printed to produce bio-printed kidney tissue. In an example, cells are cultured in a cell culture medium comprising FGF. For example, cells are cultured in a cell culture medium comprising an above referenced level of FGF9, FGF2 or FGF20 after dissociation and/or bio-printing. In an example, cells are cultured in a cell culture medium comprising 100 ng/ml FGF9 after dissociation and/or bio-printing. In another example, cells are cultured in a cell culture medium comprising 200 ng/ml FGF9 after dissociation and/or bio-printing. In these examples, the cell culture medium can also comprise heparin. For example, the cell culture medium can comprise FGF9 and 1 μg/ml heparin after dissociation and/or bio-printing. In these examples, cells can be cultured in cell culture medium comprising FGF and heparin for 4 to 6 days after dissociation and/or bio-printing. In an example, cells can be cultured in cell culture medium comprising FGF and heparin for 5 days after dissociation and/or bio-printing.

In an example, FGF is removed from the cell culture medium 4 to 6 days after dissociation and/or bio-printing. In another example, FGF is removed from the cell culture medium 5 days after dissociation and/or bio-printing. In an example, no growth factors are provided in the culture medium 5 days after dissociation and/or bio-printing.

In an example, the cell culture medium used after dissociation and/or bio-printing can also comprise retinoic acid. In an example, all trans retinoic acid (atRA) is added to cell culture medium after dissociation and/or bio-printing. In an example, at least 0.07 μM retinoic acid is added to the cell culture medium. In an example, at least 0.1 μM retinoic acid is added to the cell culture medium. In an example, at least 0.2 μM retinoic acid is added to the cell culture medium. In an example, at least 0.5 μM retinoic acid is added to the cell culture medium.

In another example, at least 1.5 μM retinoic acid is added to the cell culture medium. In an example, at least 1.8 μM retinoic acid is added to the cell culture medium. In an example, at least 2.0 μM retinoic acid is added to the cell culture medium. In another example, at least 2.5 μM retinoic acid is added to the cell culture medium. In another example, between 1.5 μM and 10 μM retinoic acid is added to the cell culture medium. In another example, between 1.5 μM and 5 μM retinoic acid is added to the cell culture medium. In another example, between 2.0 μM and 8 μM retinoic acid is added to the cell culture medium. In another example, between 2.0 μM and 3 μM retinoic acid is added to the cell culture medium.

In an example, retinoic acid is added to the cell culture medium 4 days after dissociation and/or bio-printing. In another example, retinoic acid is added to the cell culture medium 5 days after dissociation and/or bio-printing. In another example, retinoic acid is added to the cell culture medium 4 to 6 days after dissociation and/or bio-printing.

Bio-printed kidney tissue encompassed by the present disclosure and produced according to the methods disclosed herein can be described based on number of days in culture. The days in culture can be separated into two components including days for production of IM cells from stem cells (X) and days for formation of kidney tissue from (bio-printed) IM cells (Y). In an example, the step distinguishing production of IM cells from stem cells and production of bio-printed kidney tissue from IM cells is the dissociation of IM cells. One way of representing the days in culture for production of IM cells from stem cells and days for formation of bio-printed kidney tissue from IM cells is day (d) X+Y (e.g. d7+12 would describe 7 days of producing IM cells from stem cells followed by dissociation and bio-printing of IM cells and 12 days of “induction” of kidney tissue formation from IM cells (i.e. Y=number of days as bio-printed kidney tissue in culture).

In an example, the days in culture can be 7 days for production of IM cells from stem cells and from 4 days to 30 days or more for formation of kidney tissue from (bio-printed) IM cells (d7+4 to d7+30, where the day of printing is d7+0). In an example, the bio-printed kidney tissue is d7+8 to d7+20 kidney tissue. In an example, the bio-printed kidney tissue is d7+10 to d7+15 kidney tissue. In an example, the bio-printed kidney tissue is d7+12 kidney tissue. In another example, the bio-printed kidney tissue is d7+14 kidney tissue. In another example, the bio-printed kidney tissue is d7+15 kidney tissue. In another example, the bio-printed kidney tissue is d7+16 kidney tissue. In another example, the bio-printed kidney tissue is d7+17 kidney tissue. In another example, the bio-printed kidney tissue is d7+18 kidney tissue. In another example, the bio-printed kidney tissue is d7+19 kidney tissue. In another example, the bio-printed kidney tissue is d7+20 kidney tissue. In another example, the bio-printed kidney tissue is d7+21 kidney tissue. In another example, the bio-printed kidney tissue is d7+22 kidney tissue. In another example, the bio-printed kidney tissue is d7+23 kidney tissue. In another example, the bio-printed kidney tissue is d7+24 kidney tissue. In another example, the bio-printed kidney tissue is d7+25 kidney tissue.

In another example, the bio-printed kidney tissue is d7+30 kidney tissue. In another example, the bio-printed kidney tissue is between d7+12 and d7+30. In the above referenced examples stem cells may be cultured for about 8, 9, 10, 11, 12, 13 or 14 days up to about 28 days (i.e. d8+Y, d9+Y, d10+Y, d11+Y, d12+Y, d13+Y or d14+Y up to about d28+Y).

According to another embodiment, the bio-printed kidney tissue comprises from about 2 to about 100 nephrons/10,000 cells printed. In an embodiment, the bio-printed kidney tissue comprises from about 2 to about 50 nephrons/10,000 cells printed. In an embodiment, the bio-printed kidney tissue comprises from about 2 to about 45 nephrons/10,000 cells printed. In an embodiment, the bio-printed kidney tissue comprises from about 5 to about 30 nephrons/10,000 cells printed. In an embodiment, the bio-printed kidney tissue comprises from about 5 to about 20 nephrons/10,000 cells printed. In an embodiment, the bio-printed kidney tissue comprises from about 5 to about 10 nephrons/10,000 cells printed. In an embodiment, bio-printed kidney tissue is characterised in terms of % nephron, % stroma and/or % vasculature. “Nephrons” are the functional working units of kidney which play a major role in removal of waste products and maintenance of body fluid volume. They can be identified and counted in bio-printed kidney tissue disclosed herein by those of skill in the art using various methods. For example, nephrons can be visualized and counted using confocal microscopy and immunofluorescence labelling (e.g. WT1+ glomerulus; MAFB+NPHS1+ podocytes, HNF4A+LTL+ECAD− proximal tubule, SLC12A1+ECAD+ distal tubule and ECAD+GATA3+ collecting duct). In this embodiment, bio-printed kidney tissue can be additionally or alternatively characterized using single cell RNA sequencing, PCR based gene expression analysis, or immunohistochemical methods.

In one embodiment the bio-printed kidney tissue comprises a surface area of nephron tissue of greater than 0.2 mm² per 10,000 cells printed. In an embodiment, the bio-printed kidney tissue comprises a surface area of nephron tissue of 0.2 mm² to 1.5 mm² per 10,000 cells printed. In an embodiment, the bio-printed kidney tissue comprises a surface area of nephron tissue of 0.25 mm², 0.3 mm², 0.4 mm², 0.5 mm², 0.6 mm², 0.7 mm², 0.8 mm², 0.9 mm², 1 mm², 1.1 mm², 1.2 mm², 1.3 mm², 1.4 mm², or 1.5 mm² per 10,000 cells printed. In an embodiment, the bio-printed kidney tissue comprises a surface area of cells which express MAFB of greater than 0.2 mm² per 10,000 cells printed. In an embodiment, the bio-printed kidney tissue comprises a surface area of cells which express MAFB of 0.2 mm² to 1.5 mm² per 10,000 cells printed. In an embodiment, the bio-printed kidney tissue comprises a surface area of cells which express MAFB of 0.25 mm², 0.3 mm², 0.4 mm², 0.5 mm², 0.6 mm², 0.7 mm², 0.8 mm², 0.9 mm², 1 mm², 1.1 mm², 1.2 mm², 1.3 mm², 1.4 mm², or 1.5 mm² per 10,000 cells printed.

In one embodiment, the bio-printed kidney tissue has an even distribution of nephrons across the bio-printed layer. That is, in contrast to manually aggregated organoids or bio-printed kidney organoids of a suboptimal confirmation generated as a dot or a blob of cells as described in the prior art and which form domed structures of a height >150 uM from the Transwell and having unpatterned central areas or cores lacking nephrons. This embodiment describes a bio-printed kidney tissue comprising a larger number and more uniform distribution of nephrons with no core of non-nephron tissue. For example, the bio-printed kidney has an even distribution of glomeruli, as marked by e.g. cells expressing MAFB, across the bio-printed layer. In another embodiment, the bio-printed kidney tissue expresses of one or more of SLC12A1, CDH1, HNF4A, CUBN, LRP2, EPCAM and MAFB across the entire structure. In another embodiment, the bio-printed kidney tissue shows an increased expression or high levels of one or more of SLC12A1, CDH1, HNF4A, CUBN, LRP2, EPCAM and MAFB compared to a kidney organoid prepared according to previously published methodologies (Takasato et al. (2015) Nature, Vol. 526:564-568) i.e. manually aggregated, or bio-printed kidney organoids generated as a dot or a blob of cells. In another embodiment, the bio-printed kidney tissue shows an increased expression or high levels of one or more of SLC30A1, SLC51B, FABP3, and SULT1E1 (genes associated with proximal tubule maturity) and/or decreased expression of either or both of SPP1, JAG1 (genes associated with early immature tubule) compared to a kidney organoid prepared according to previously published methodologies (Takasato et al. (2015) Nature, Vol. 526:564-568) i.e. manually aggregated, or bio-printed kidney organoids generated as a dot or a blob of cells. In one embodiment, the bio-printed kidney tissue shows low to no expression of one or more of THY1, DCN, SOX17, FLT1 and PECAM, or decreased expression of one or more of THY1, DCN, SOX17, FLT1 and PECAM compared to a kidney organoid prepared according to previously published methodologies (Takasato et al. (2015) Nature, Vol. 526:564-568) i.e. manually aggregated, or bio-printed kidney organoids generated as a dot or a blob of cells. That is, in the above examples, high and low levels of expression are relative to kidney organoids cultured via the method described in Takasato et al. (2015) Nature, Vol. 526:564-568, Takasato et al. (2016) Nat Protocols, 11:1681-1692, or Takasato et al. (2014) Nat. Cell Biol., 16:118-127. In this example, high expression is at least 1.5-fold higher. In another example, high expression is at least 2-fold higher. In another example, high expression is at least 3-fold higher. In an example, low expression is at least 1.5-fold lower. In another example, low expression is at least 2-fold lower. In another example, low expression is at least 3-fold lower.

Expression levels can be measured using techniques such as polymerase chain reaction comprising appropriate primers for markers of interest. For example, total RNA can be extracted from cells before being reverse transcribed and subject to PCR and analysis.

The inventors have also surprisingly found that in “non-nephron” tissue in the bio-printed kidney tissue shows an increased expression or genes associated with nephron progenitor identity compared to a kidney organoid prepared according to previously published methodologies (Takasato et al. (2015) Nature, Vol. 526:564-568) i.e. manually aggregated, or bio-printed kidney organoids generated as a dot or a blob of cells. In another embodiment, the bio-printed kidney tissue shows an increased expression or high levels of one or more of HOXA11, FOXC2, EYA1, and SIX2 compared to a kidney organoid prepared according to previously published methodologies (Takasato et al. (2015) Nature, Vol. 526:564-568) i.e. manually aggregated, or bio-printed kidney organoids generated as a dot or a blob of cells.)

In another embodiment, the bio-printed kidney tissue further comprises a bio-compatible scaffold. For example, in another embodiment, the bio-ink is bio-printed onto a bio-compatible scaffold. That is the surface onto which the bio-ink is printed is a biocompatible scaffold. In one embodiment, the biocompatible scaffold is biodegradable or bio-absorbable. In another embodiment, the biocompatible scaffold is a hydrogel. In another embodiment, the scaffold may be functionalised with one or more agents (e.g. bioactive agents). For example, the bioactive agents (such as cytokines, chemokines, differentiation factors, signalling pathway inhibitors) may, for example, facilitate the further development or differentiation of cells in the bio-ink printed thereon.

In another embodiment, the bio-ink further comprises one or more bioactive agents. In one example, the one or more bioactive agents promotes induction of kidney tissue from the plurality of cells. In another embodiment, the bio-ink further comprises differentiation media, bio-printing media, or any combination thereof. In some embodiments, the bio-printing media includes a hydrogel, including a modified hydrogel or a functionalized hydrogel, or matrix components or a mixture of extracellular matrix components. In another embodiment the bio-ink comprises hyaluronic acid. In one embodiment the one or more agents is selected from the group consisting of: anti-proliferative agents, immunosuppressants, pro-angiogenic compounds, antibodies or fragments or portions thereof, antibiotics or antimicrobial compounds, antigens or epitopes, aptamers, biopolymers, carbohydrates, cell attachment mediators (such as RGD), cytokines, cytotoxic agents, drugs, enzymes, growth factors or recombinant growth factors and fragments and variants thereof, hormone antagonists, hormones, immunological agents, lipids, metals, nanoparticles, nucleic acid analogs, nucleic acids (e.g., DNA, RNA, siRNA, RNAi, and microRNA agents), nucleotides, nutraceutical agents, oligonucleotides, peptide nucleic acids (PNA), peptides, prodrugs, prophylactic agents, proteins, small molecules, therapeutic agents, or any combinations thereof.

In another embodiment, the bio-printed kidney tissue further comprises a bio-ink as described herein above which is positioned adjacent or in close proximity to another bio-printed bio-ink which may optionally contain one or more agents as described above, or one or more other cell types. For example, the bio-ink comprising a plurality of cells (and optionally one or more agents) may be bio-printed so as to abut, or be in close proximity to, another bio-printed bio-ink. For example, a bio-ink comprising a plurality of cells (and optionally one or more agents) may be bio-printed on top of, or next to, including directly onto or next to, a line or layer of bio-printed bio-ink (optionally comprising one or more agents and/or one or more other cell types). For example, the method for producing bio-printed kidney tissue comprises bio-printing a pre-determined amount of a first bio-ink and printing a pre-determined amount of a second bio-ink onto a surface, wherein the first bio-ink and the second bio-ink are different. In one example, the first bio-ink contains a plurality of cells that are different to the plurality of cells in the second bio-ink. In another example, the first bio-ink contains a plurality of cells, while the second bio-ink does not contain cells but may contain other ingredients, such as for example, a bio-active agent.

### Methods for Producing Bio-Printed Kidney Tissue

In another aspect, the present invention relates to methods for the production of bio-printed kidney tissue. In one embodiment, the method for producing bio-printed kidney tissue comprises the steps of: bio-printing a pre-determined amount of a bio-ink onto a surface, wherein the bio-ink comprises a plurality of cells, and wherein the bio-ink is bio-printed in a layer that is less than about 150 μm high; and inducing the bio-printed, pre-determined amount of the bio-ink to form bio-printed kidney tissue. Preferably, the bio-ink is bio-printed in a layer that is about 50 μm high or less.

In one embodiment, at the step of bio-printing, the bio-ink comprising a plurality of cells is bio-printed in a layer that is less than about 150 μm high. In one embodiment, the bio-ink is bio-printed in a layer selected from about 15 μm to about 150 μm. In one embodiment, the bio-ink is bio-printed in a layer selected from about 25 μm high to about 100 μm high. In a preferred embodiment, the bio-ink is bio-printed in a layer about 50 μm high or less. In one embodiment, the bio-ink is bio-printed in a layer about 15 μm high. In one embodiment, the bio-ink is bio-printed in a layer about 20 μm high. In one embodiment, the bio-ink is bio-printed in a layer about 25 μm high. In one embodiment, the bio-ink is bio-printed in a layer about 30 μm high. In one embodiment, the bio-ink is bio-printed in a layer about 35 μm high. In one embodiment, the bio-ink is bio-printed in a layer about 40 μm high. In one embodiment, the bio-ink is bio-printed in a layer about 50 μm high. In one embodiment, the bio-ink is bio-printed in a layer about 60 μm high. In one embodiment, the bio-ink is bio-printed in a layer about 70 μm high. In one embodiment, the bio-ink is bio-printed in a layer about 80 μm high. In one embodiment, the bio-ink is bio-printed in a layer about 90 μm high. In one embodiment, the bio-ink is bio-printed in a layer about 100 μm high.

In one embodiment, at the bio-printing step, the bio-printed layer of bio-ink comprises between about 5,000 and about 100,000 cells per mm². In one embodiment, the bio-printed layer of bio-ink comprises between about 10,000 and about 50,000 cells per mm². In one embodiment, the bio-printed layer of bio-ink comprises between about 5,000 and about 50,000 cells per mm². In one embodiment, the bio-printed layer of bio-ink comprises between about 10,000 and about 40,000 cells per mm². In one embodiment, the bio-printed layer of bio-ink comprises between about 10,000 and about 30,000 cells per mm². In one embodiment, the bio-printed layer of bio-ink comprises from about 10,000 to about 20,000 cells per mm². In one embodiment, the bio-printed layer of bio-ink comprises about 30,000 cells per mm² or less. In one embodiment, the bio-printed layer of bio-ink comprises about 5,000 cells per mm². In one embodiment, the bio-printed layer of bio-ink comprises about 10,000 cells per mm². In one embodiment, the bio-printed layer of bio-ink comprises about 15,000 cells per mm². In one embodiment, the bio-printed layer of bio-ink comprises about 20,000 cells per mm². In one embodiment, the bio-printed layer of bio-ink comprises about 30,000 cells per mm². In one embodiment, the bio-printed layer of bio-ink comprises about 40,000 cells per mm². In one embodiment, the bio-printed layer of bio-ink comprises about 50,000 cells per mm². In one embodiment, the bio-printed layer of bio-ink comprises about 60,000 cells per mm². In one embodiment, the bio-printed layer of bio-ink comprises about 70,000 cells per mm². In one embodiment, the bio-printed layer of bio-ink comprises about 80,000 cells per mm². In one embodiment, the bio-printed layer of bio-ink comprises about 90,000 cells per mm². In one embodiment, the bio-printed layer of bio-ink comprises about 100,000 cells per mm².

According to a preferred embodiment, at the bio-printing step, the bio-printed layer of bio-ink comprises from about 10,000 cells to about 20,000 cells per mm² and having a height of about 50 μm or less. In a further preferred embodiment, at the bio-printing step, the bio-printed layer of bio-ink comprises a bio-printed layer of bio-ink comprising about 20,000 cells per mm² and having a height of about 40 μm or less. In a further preferred embodiment, at the bio-printing step, the bio-printed layer of bio-ink comprises about 14,000 cells per mm² and having a height of about 30 μm or less. In a further preferred embodiment, at the bio-printing step, the bio-printed layer of bio-ink comprises about 11,000 cells per mm² and having a height of about 25 μm or less. In a further preferred embodiment, at the bio-printing step, the bio-printed layer of bio-ink comprises about 10,000 cells per mm² and having a height of about 20 μm or less.

In a preferred embodiment, the bio-ink is a wet cell paste. In another embodiment, at the bio-printing step, the bio-ink comprises between approximately 10,000 cells/μl and approximately 400,000 cells/μl. In one embodiment, the bio-ink comprises between about 10,000 cells/μl and about 100,000 cells/μl. In one embodiment, the bio-ink comprises between about 100,000 cells/μl and about 400,000 cells/μl. In one embodiment, the bio-ink comprises between about 50,000 cells/μl and about 200,000 cells/μl. In one embodiment, the bio-ink comprises about 10,000 cells/μl, about 30,000 cells/μl, about 40,000 cells/μl, about 50,000 cells/μl, about 60,000 cells/μl, about 70,000 cells/μl, about 80,000 cells/μl, about 90,000 cells/μl, about 100,000 cells/μl, about 150,000 cells/μl, about 200,000 cells/μl, about 250,000 cells/μl, about 300,000 cells/μl, or about 400,000 cells/μl. In a preferred embodiment, the bio-ink comprises about 200,000 cells/μl.

In some embodiments, the bio-ink comprises partly differentiated cells. In some embodiments, the bio-ink comprises fully differentiated cells.

In some embodiments, the bio-ink comprises cells differentiated from human stem cells (HSCs), including but not limited to, human induced pluripotent stem cells (iPSCs) and human embryonic stem cells (hESCs). In some embodiments, the bio-ink comprises primitive streak cells, including but not limited to posterior primitive streak cells. In some embodiments, the bio-ink comprises intermediate mesoderm (IM) cells. In some embodiments, the bio-ink comprises metanephric mesenchyme (MM) cells. In some embodiments, the bio-ink comprises nephric duct cells. In some embodiments, the bio-ink comprises renal progenitor cells, including but not limited to nephron progenitor cells, ureteric epithelial progenitor cells, or a combination thereof.

In some embodiments, the cells of the bio-ink comprise patient-derived cells. In some embodiments, the cells of the bio-ink comprise gene-edited cells. In some embodiments, the cells of the bio-ink comprise patient-derived cells that are also gene-edited cells. In some embodiments, the cells of the bio-ink comprise cells from a reporter line. In some embodiments, the cells of the bio-ink comprise a reporter line cell that is also gene edited.

Through employing the methods for producing bio-printed kidney tissue disclosed herein, a bio-printed engineered kidney tissue which is enriched for nephrons can be produced. According to another embodiment, the bio-printed kidney tissue prepared according to the methods described and exemplified herein comprises from about 2 to about 100 nephrons/10,000 cells printed. According to another embodiment, the bio-printed kidney tissue prepared according to the methods described and exemplified herein comprises from about 2 to about 50 nephrons/10,000 cells printed. According to another embodiment, the bio-printed kidney tissue prepared according to the methods described and exemplified herein comprises from about 5 to about 40 nephrons/10,000 cells printed. According to another embodiment, the bio-printed kidney tissue prepared according to the methods described and exemplified herein comprises from about 5 to about 75 nephrons/10,000 cells printed. According to another embodiment, the bio-printed kidney tissue prepared according to the methods described and exemplified herein comprises from about 5 to about 60 nephrons/10,000 cells printed. According to another embodiment, the bio-printed kidney tissue prepared according to the methods described and exemplified herein comprises from about 5 to about 50 nephrons/10,000 cells printed. According to another embodiment, the bio-printed kidney tissue prepared according to the methods described and exemplified herein comprises from about 5 to about 40 nephrons/10,000 cells printed. In an embodiment, the bio-printed kidney tissue comprises from about 5 to about 20 nephrons/10,000 cells printed. In an embodiment, the bio-printed kidney tissue comprises from about 5 to about 10 nephrons/10,000 cells printed. As detailed herein, nephrons can be identified and counted in bio-printed kidney tissue disclosed herein by those of skill in the art using various methods including visualization and counting using confocal microscopy and immunofluorescence labelling (e.g. for WT1+ glomerulus; MAFB+NPHS1+ podocytes, HNF4A+LTL+ECAD− proximal tubule, SLC12A1+ECAD+ distal tubule and ECAD+GATA3+ connecting segment or collecting duct). In this embodiment, bio-printed kidney tissue can be additionally or alternatively characterized using single cell RNA sequencing, PCR based gene expression analysis, immunofluorescence labelling or immunohistochemical methods. In one embodiment the bio-printed kidney tissue comprises a surface area of nephron tissue of greater than 0.2 mm² per 10,000 cells printed. In an embodiment, the bio-printed kidney tissue comprises a surface area of nephron tissue of 0.2 mm² to 1.5 mm² per 10,000 cells printed. In an embodiment, the bio-printed kidney tissue comprises a surface area of nephron tissue of 0.25 mm², 0.3 mm², 0.4 mm², 0.5 mm², 0.6 mm², 0.7 mm², 0.8 mm², 0.9 mm², 1 mm², 1.1 mm², 1.2 mm², 1.3 mm², 1.4 mm², or 1.5 mm² per 10,000 cells printed. In an embodiment, the bio-printed kidney tissue comprises a surface area of cells which express MAFB of greater than 0.2 mm² per 10,000 cells printed. In an embodiment, the bio-printed kidney tissue comprises a surface area of cells which express MAFB of 0.2 mm² to 1.5 mm² per 10,000 cells printed. In an embodiment, the bio-printed kidney tissue comprises a surface area of cells which express MAFB of 0.25 mm², 0.3 mm², 0.4 mm², 0.5 mm², 0.6 mm², 0.7 mm², 0.8 mm², 0.9 mm², 1 mm², 1.1 mm², 1.2 mm², 1.3 mm², 1.4 mm², or 1.5 mm² per 10,000 cells printed.

According to another embodiment, the bio-printed kidney tissue produced according to the methods disclosed herein has an even distribution of nephrons across the bio-printed layer. That is, in contrast to manually aggregated or bio-printed kidney organoids which can be generated as a dot or a blob of cells as described in the prior art and which form domed structures having stromal centres lacking nephrons, the bio-printed kidney tissue comprises a larger number and more uniform distribution of nephrons. For example, the bio-printed kidney has an even distribution of glomeruli, as marked by e.g. cells expressing MAFB, across the bio-printed layer. In another embodiment, the bio-printed kidney tissue expresses of one or more of SLC12A1, CDH1, HNF4A, CUBN, LRP2, EPCAM and MAFB. In another embodiment, the bio-printed kidney tissue shows an increased expression of one or more of SLC12A1, CDH1, HNF4A, CUBN, LRP2, EPCAM and MAFB compared to a kidney organoid prepared according to previously published methodologies (Takasato et al. (2015) Nature, Vol. 526:564-568) i.e. manually aggregated, or bio-printed kidney organoids generated as a dot or a blob of cells. In one embodiment, the bio-printed kidney tissue shows low to no expression of one or more of THY1, DCN, SOX17, FLT1 and PECAM, or decreased expression of one or more of THY1, DCN, SOX17, FLT1 and PECAM compared to a kidney organoid prepared according to previously published methodologies (Takasato et al. (2015) Nature, Vol. 526:564-568) i.e. manually aggregated, or bio-printed kidney organoids generated as a dot or a blob of cells. In another embodiment, the bio-printed kidney tissue has nephrons in which the proximal tubule and distal tubule segments shows markers of maturation, including HNF4A and SLC12A1. In another embodiment, the bio-printed kidney tissue shows reduced presence of stroma, fibroblasts and endothelial cells. In another embodiment, the bio-printed kidney tissue shows reduced off target populations with respect to nephron cell types.

In another embodiment, at the step of bio-printing, the bio-ink is bio-printed onto a bio-compatible scaffold. That is the surface onto which the bio-ink is printed is a biocompatible scaffold. In one embodiment, the biocompatible scaffold is biodegradable or bio-absorbable. In another embodiment, the biocompatible scaffold is a hydrogel. In another embodiment, the scaffold may be functionalised with one or more bioactive agents. For example, the bioactive agents (e.g. small molecules, polypeptides including cytokines and chemokines, differentiation factors, signalling pathway inhibitors etc.) may, for example, facilitate viability of the cells in the bio-ink and the further development or differentiation of cells in the bio-ink.

In another embodiment, the bio-ink further comprises one or more agents (e.g. bioactive agents). In one example, the one or more bioactive agents promotes induction of kidney tissue from the plurality of cells. In another embodiment, the bio-ink further comprises differentiation media, bio-printing media, or any combination thereof. In some embodiments, the bio-printing media includes a hydrogel and/or one or more ECM components. In one embodiment, the bio-ink comprises hyaluronic acid. In one embodiment the one or more agents is selected from the group consisting of: anti-proliferative agents, immunosuppressants, pro-angiogenic compounds, antibodies or fragments or portions thereof, antibiotics or antimicrobial compounds, antigens or epitopes, aptamers, biopolymers, carbohydrates, cell attachment mediators (such as RGD), cytokines, cytotoxic agents, drugs, enzymes, growth factors or recombinant growth factors and fragments and variants thereof, hormone antagonists, hormones, immunological agents, lipids, metals, nanoparticles, nucleic acid analogs, nucleic acids (e.g., DNA, RNA, siRNA, RNAi, and microRNA agents), nucleotides, nutraceutical agents, oligonucleotides, peptide nucleic acids (PNA), peptides, prodrugs, prophylactic agents, proteins, small molecules, therapeutic agents, or any combinations thereof.

In another embodiment, the bio-printed kidney tissue further comprises a bio-ink as described herein above which is positioned adjacent or in close proximity to another bio-printed bio-ink which may optionally contain one or more agents as described above, or one or more other cell types. For example, the bio-ink comprising a plurality of cells (and optionally one or more agents) may be bio-printed so as to abut, or be in close proximity to, another bio-printed bio-ink. For example, a bio-ink comprising a plurality of cells (and optionally one or more agents) may be bio-printed on top of, or next to, including directly onto or next to, a line or layer of bio-printed bio-ink (optionally comprising one or more agents and/or one or more other cell types). In one embodiment, the method for producing bio-printed kidney tissue comprises bio-printing a pre-determined amount of a first bio-ink and printing a pre-determined amount of a second bio-ink onto a surface, wherein the first bio-ink and the second bio-ink are different. In one example, the first bio-ink contains a plurality of cells that are different to the plurality of cells in the second bio-ink. In another example, the first bio-ink contains a plurality of cells, while the second bio-ink does not contain cells but may contain other ingredients, such as for example, a bio-active agent.

In an example, the step of inducing the bio-printed bio-ink to form kidney tissue comprises contacting the bio-ink with FGF-9. In another example, the bio-ink is induced to form kidney tissue by contacting the bio-ink with FGF-9 for a period of 5 days. In some examples, the plurality of cells may be briefly contacted with a cell culture medium comprising CHIR before being bio-printed and further cultured. For example, the plurality of cells can be contacted with a cell culture medium comprising 3 to 8 μM CHIR for one to two hours before being bio-printed and further cultured. In another example, plurality of cells can be contacted with a cell culture medium comprising 5 μM CHIR for one hour before being bio-printed and further cultured. In another embodiment the step of inducing the bio-printed bio-ink to form kidney tissue comprises briefly contacting the bio-ink with a cell culture medium comprising CHIR after being bio-printed and further cultured. In one embodiment, the method comprises culturing the bio-printed bio-ink for 1 hour in the presence of 5 to 10 μM CHIR.

In one embodiment, the plurality of cells comprises a culture expanded population of stem cell-derived intermediate mesoderm (IM) cells. The IM cells can be prepared and cultured according to the methods described in the section entitled “Bio-Printed Kidney Tissue” above. In one embodiment, the step of inducing comprises contacting the bio-printed, predetermined amount of bio-ink with FGF-9. In another embodiment, the step of inducing comprises contacting the bio-printed, predetermined amount of bio-ink with FGF-9 for a period of 5 days. In one embodiment, the step of inducing the bio-printed, pre-determined amount of the bio-ink to form bio-printed kidney tissue is performed as described in the section entitled “Bio-Printed Kidney Tissue” above.

Extrusion bio-printing allows control over cell aggregate shape, cell number, cell density and final tissue height (or thickness) by introducing fine tip movement as cells are extruded. Via scripting of the movement of the extrusion port during the process of extrusion, the bio-ink can be spread over a defined distance in a way that would not be possible to control, or at least reproduce with accuracy, manually. Increasing the amount of tip movement for a given rate of cell extrusion (ratio) enables the user to create bio-printed tissue of variable cell density, shape and height (thickness) as cells are spread, and subsequently aggregate, over larger surface areas. According to one embodiment, the bio-printing step uses an extrusion-based bio-printer. In another embodiment, the bio-printing step uses an extrusion-based bio-printer with a syringe of 100-500 μl and a needle with an internal diameter of between about 100 to about 550 μm.

In one embodiment, at the step of bio-printing, a dispensing apparatus of a bio-printer is configured to dispense said layer in one or more lines. In another embodiment, at the step of bio-printing, a dispensing apparatus of a bio-printer is configured to dispense said layer in one or more lines so as to form a continuous sheet or patch.

An extrusion bio-printer to be employed in the methods disclosed herein can be scripted to regulate the speed of extrusion of the bio-ink with the movement of the dispensing apparatus. This is referred to as the ‘ratio’. For example, this term refers to the rate of material dispensed across a certain degree of movement of tip through which the bio-ink is extruded. A high ratio refers to more tip movement for the same amount of extrusion. Increasing or decreasing dispense ratio increases or decreases area across which a certain amount of bio-ink volume is extruded. Hence, ratio could be defined as cells/mm tip movement. In one embodiment, the ratio of 40, 30, 20 or 10 would be equivalent to about 9,000 cells/mm, about 12,000 cells/mm, about 18,000 cells/mm and about 36,000 cells/mm where mm is mm of tip movement, preferably wherein the tip is of a 25G needle.

The height of the bio-printed layer of a predetermined amount of a bio-ink at printing will decrease as the dispense ratio increases. That is, the height of the bio-printed layer of a predetermined amount of a bio-ink at printing declines with line length. In a preferred embodiment, the height of the bio-printed layer of bio-ink is about 50 μm or less. The height of the same bio-printed structures after differentiation (e.g. after the “inducing” step in the methods described herein) can vary depending upon the number of days of culture. Examples provided here present tissue structures cultured for a further 12 days, during which the printed layer of bio-ink undergoes self-organisation of the component cells and differentiation into differentiated cell types. In a preferred embodiment, the height of the bio-printed tissue is about 150 μm or less after a period of time in culture.

In a preferred embodiment, the method for the method for producing bio-printed kidney tissue comprises the steps of: i) bio-printing an amount of a bio-ink comprising a plurality of cells onto a surface to produce a layer of said bio-ink, wherein the height of the layer of bio-ink is about 50 μm or less and comprises from about 10,000 cells to about 20,000 cells per mm², and wherein the cells are stem cell-derived IM cells; and ii) inducing the printed bio-ink to form kidney tissue.

According to another aspect, the present invention provides bio-printed kidney tissue produced according to the methods described herein.

### Tissue Engineering of Kidney Tissue for Transplantation

To engineer human kidney tissue for the purposes of transplantation into kidney disease and renal failure patients, there is a need to increase the number of nephrons forming per engineered structure and per starting cell type and create a biocompatible structure amendable for transplantation under the renal capsule. Manually generated organoids or bio-printed dots can be vascularized by a recipient animal when transplanted under the renal capsule. However, problems associated with transplantation of such engineered tissue is ‘off target’ tissue differentiation and stromal overgrowth. Accordingly, a better tissue for transplantation is required.

As described herein, the present inventors have also surprisingly identified that the bio-printed kidney tissue disclosed herein has a high nephron content. Without wishing to be bound by any particular theory, an increased number of nephrons forming per structure and per starting cell type, may create a biocompatible structure amendable for transplantation under the renal capsule. These features may indicate that bio-printed kidney tissue is more suitable for therapeutic applications such as transplantation. For example, the bio-printed kidney tissue may avoid the problem of off target tissue differentiation and stromal overgrowth. The bio-printed kidney tissue defined herein may represent a better tissue for transplantation.

According to one aspect, the present invention relates to bio-printed kidney tissue disclosed herein or produced according to the methods disclosed herein for use in the treatment of kidney disease or renal failure in a subject in need thereof. As such, the present invention also relates to the use of bio-printed kidney tissue disclosed herein or produced according to the methods disclosed herein for use in transplantation into a kidney disease or renal failure patient.

The present invention also relates to methods of treatment of kidney disease or renal failure in patient in need thereof comprising administering to the patient bio-printed kidney tissue disclosed herein or produced according to the methods disclosed herein. In one embodiment, the bio-printed kidney tissue is enriched with nephrons distributed throughout the tissue. This is in contrast to a bio-printed kidney organoid where fewer nephrons are produced and are only distributed around the periphery of the organoid.

In one embodiment bio-printed kidney tissue comprises a bio-ink, wherein the bio-ink comprises a plurality of cells, and wherein the bio-printed kidney tissue comprises a surface area of nephron tissue of greater than 0.2 mm² per 10,000 cells printed. In an embodiment, the bio-printed kidney tissue comprises a surface area of nephron tissue of 0.2 mm² to 1.5 mm² per 10,000 cells printed. In an embodiment, the bio-printed kidney tissue comprises a surface area of nephron tissue of 0.25 mm², 0.3 mm², 0.4 mm², 0.5 mm², 0.6 mm², 0.7 mm², 0.8 mm², 0.9 mm², 1 mm², 1.1 mm², 1.2 mm², 1.3 mm², 1.4 mm², or 1.5 mm² per 10,000 cells printed. In an embodiment, the bio-printed kidney tissue comprises a surface area of cells which express MAFB of greater than 0.2 mm² per 10,000 cells printed. In an embodiment, the bio-printed kidney tissue comprises a surface area of cells which express MAFB of 0.2 mm² to 1.5 mm² per 10,000 cells printed. In an embodiment, the bio-printed kidney tissue comprises a surface area of cells which express MAFB of 0.25 mm², 0.3 mm², 0.4 mm², 0.5 mm², 0.6 mm², 0.7 mm², 0.8 mm², 0.9 mm², 1 mm², 1.1 mm², 1.2 mm², 1.3 mm², 1.4 mm², or 1.5 mm² per 10,000 cells printed.

In an example, the bio-printed kidney tissue comprises from about 5 to about 100 nephrons/mm² of bio-printed kidney tissue. In an example, the bio-printed kidney tissue comprises from about 5 to about 75 nephrons/mm² of bio-printed kidney tissue. In an example, the bio-printed kidney tissue comprises from about 5 to about 50 nephrons/mm² of bio-printed kidney tissue. In an example, the bio-printed kidney tissue comprises from about 5 to about 20 nephrons/mm² of bio-printed kidney tissue. In an example, the bio-printed kidney tissue comprises from about 20 to about 50 nephrons/mm² of bio-printed kidney tissue.

In an example, the bio-printed kidney tissue has an even distribution of glomeruli, as marked by e.g. cells expressing MAFB, across the bio-printed layer. In another embodiment, the bio-printed kidney tissue expresses of one or more of SLC12A1, CDH1, HNF4A, CUBN, LRP2, EPCAM and MAFB. In another embodiment, the bio-printed kidney tissue shows an increased expression of one or more of SLC12A1, CDH1, HNF4A, CUBN, LRP2, EPCAM and MAFB compared to a kidney organoid prepared according to previously published methodologies (Takasato et al. (2015) Nature, Vol. 526:564-568) i.e. manually aggregated, or bio-printed kidney organoids generated as a dot or a blob of cells. In one embodiment, the bio-printed kidney tissue shows low to no expression of one or more of THY1, DCN, SOX17, FLT1 and PECAM, or decreased expression of one or more of THY1, DCN, SOX17, FLT1 and PECAM compared to a kidney organoid prepared according to previously published methodologies (Takasato et al. (2015) Nature, Vol. 526:564-568) i.e. manually aggregated, or bio-printed kidney organoids generated as a dot or a blob of cells. In another embodiment, the bio-printed kidney tissue has nephrons in which the proximal tubule and distal tubule segments shows markers of maturation, including HNF4A and SLC12A1. In another embodiment, the bio-printed kidney tissue shows reduced presence of stroma, fibroblasts and endothelial cells.

The bio-printed kidney tissue may be produced in a range of dimensions suitable for transplantation. In some embodiments, the bio-printed kidney tissue is printed at a height of from about 15 μm to about 150 μm. In one embodiment, the bio-ink is bio-printed in a layer selected from about 25 μm high to about 100 μm high. In a preferred embodiment the bio-ink is bio-printed in a layer about 50 μm high or less. In one embodiment, the bio-ink is bio-printed in a layer about 15 μm high. In one embodiment, the bio-ink is bio-printed in a layer about 20 μm high. In one embodiment, the bio-ink is bio-printed in a layer about 25 μm high. In one embodiment, the bio-ink is bio-printed in a layer about 30 μm high. In one embodiment, the bio-ink is bio-printed in a layer about 35 μm high. In one embodiment, the bio-ink is bio-printed in a layer about 40 μm high. In one embodiment, the bio-ink is bio-printed in a layer about 50 μm high. In one embodiment, the bio-ink is bio-printed in a layer about 60 μm high. In one embodiment, the bio-ink is bio-printed in a layer about 70 μm high. In one embodiment, the bio-ink is bio-printed in a layer about 80 μm high. In one embodiment, the bio-ink is bio-printed in a layer about 90 μm high. In one embodiment, the bio-ink is bio-printed in a layer about 100 μm high. As described above the height of the bio-printed tissue may increase slightly following bio-printing such as during subsequent culture (e.g. during induction of the bio-printed bio-ink to form kidney tissue) and/or maintenance. In one embodiment, after a period of time in culture, the bio-printed tissue obtains a height which does not exceed 150 μm. In one embodiment, after a period of culture, the bio-printed tissue obtains a height which between about 100 μm and 150 μm. In another embodiment, the bio-printed kidney tissue has a length of from 1 mm to 30 mm and a width of from 0.5 mm to 20 mm. In another embodiment, the bio-printed kidney tissue has a length of from 5 mm to 30 mm and a width of from 0.5 mm to 2 mm. In another embodiment, the bio-printed kidney tissue has a height of up to approximately 100 μm to 250 μm. In this embodiment, the height (or thickness) is not the height at which the tissue is printed, but the height (or thickness) of the kidney tissue after the bio-printed bio-ink is induced (for example following a period time in culture).

In another embodiment, the bio-printed kidney tissue for use in treatment/transplantation further comprises a bio-compatible scaffold. For example, in another embodiment, the bio-ink is bio-printed onto a bio-compatible scaffold. That is, the surface onto which the bio-ink is printed is a biocompatible scaffold. In one embodiment, the biocompatible scaffold is biodegradable or bio-absorbable. In another embodiment, the biocompatible scaffold is a hydrogel. In another embodiment, the scaffold may be functionalized with one or more agents (e.g. bioactive agents). For example, the bioactive agents (such as cytokines, chemokines, differentiation factors, signalling pathway inhibitors) may, for example, facilitate the further development or differentiation of cells in the bio-ink printed thereon, or facilitate engraftment and/or survival of the transplanted bio-printed tissue.

In another embodiment, the bio-ink or scaffold further comprises one or more bioactive agents that promote induction of kidney tissue from the plurality of cells. In another embodiment, the bio-ink or scaffold further comprises a hydrogel, including a modified hydrogel or a functionalized hydrogel, or matrix components or a mixture of extracellular matrix components. In one embodiment the one or more agents is selected from the group consisting of: anti-proliferative agents, immunosuppressants, pro-angiogenic compounds, antibodies or fragments or portions thereof, antibiotics or antimicrobial compounds, antigens or epitopes, aptamers, biopolymers, carbohydrates, cell attachment mediators (such as RGD), cytokines, cytotoxic agents, drugs, enzymes, growth factors or recombinant growth factors and fragments and variants thereof, hormone antagonists, hormones, immunological agents, lipids, metals, nanoparticles, nucleic acid analogs, nucleic acids (e.g., DNA, RNA, siRNA, RNAi, and microRNA agents), nucleotides, nutraceutical agents, oligonucleotides, peptide nucleic acids (PNA), peptides, prodrugs, prophylactic agents, proteins, small molecules, therapeutic agents, or any combinations thereof.

According to the foregoing embodiments the bio-printed kidney tissue may be used for transplantation into a patient. This may include a patient with reduced renal function due to chronic kidney disease, inherited kidney disease or after renal reduction surgery for cancer. In one embodiment, the bio-printed tissue is transplanted under the renal capsule of a recipient. In one embodiment, the bio-printed tissue may be a sheet or a patch.

### Drug Screening

According to another aspect the present invention provides a method of screening a candidate compound for nephrotoxicity or therapeutic efficacy, the method comprising contacting the bio-printed kidney tissue as described herein with a candidate compound and determining whether or not the candidate compound is nephrotoxic or therapeutically effective.

In one embodiment, the method comprises contacting said bio-printed kidney tissue with a candidate compound and a nephrotoxin and determining whether or not the candidate compound is therapeutically effective. In one embodiment, determining whether or not the candidate compound is nephrotoxic or therapeutically effective comprises measuring one or more of: expression of one or more genes associated with cell death; expression of one or more genes associated with cell viability; expression of one or more nephron-associated genes; expression of one or more genes associated with glomerular extracellular matrix; expression of one or more genes associated with podocyte, endothelial or mesangial cell types; and intensity of expression of a reporter gene associated with at least one gene of interest.

In another embodiment, i) a measured reduction in one or more of: expression of one or more genes associated with cell viability; expression of one or more nephron-associated genes; expression of one or more genes associated with glomerular extracellular matrix; expression of one or more genes associated with podocyte, endothelial or mesangial cell types; and intensity of said reporter gene; and/or ii) a measured increase in expression of one or more genes associated with cell death; is indicative of nephrotoxicity of the candidate compound.

In another embodiment, i) a measured increase or absence of a measured reduction in one or more of: expression of one or more genes associated with cell viability; expression of one or more nephron-associated genes; expression of one or more genes associated with glomerular extracellular matrix; expression of one or more genes associated with podocyte, endothelial or mesangial cell types; and intensity of said reporter gene; and/or ii) a measured reduction in expression of one or more genes associated with cell death; is indicative of therapeutic efficacy of the candidate compound. In an embodiment the candidate compound is a small molecule, polynucleotide, peptide, protein, antibody, antibody fragment, serum, virus, bacteria, stem cell or combination thereof. In another embodiment, the candidate compound is serum including serum isolated from a subject with kidney disease.

In another embodiment, the method may further comprise selecting a candidate compound which is not nephrotoxic and/or is therapeutically effective.

### EXAMPLES

### Example 1. Human Pluripotent Stem Cell Directed Differentiation and Manual Organoid Production

Human pluripotent stem cells were thawed and seeded overnight in the presence of 1× RevitaCell (ThermoFisher Scientific catalog# A2644501), and cultured under standard feeder-free, defined conditions on GelTrex (Thermo Fisher Scientific catalog# A1413301) or Matrigel in Essential 8 medium (Thermo Fisher Scientific), with daily media changes. On the day prior to initiation of differentiation, the cells were dissociated with TrypLE Select (ThermoFisher Scientific catalog#12563011), counted using trypan exclusion on a Nexcellom Cellometer Brightfield Cell Counter (Nexcelom Biosciences), and seeded in a GelTrex, Matrigel or Laminin-521 coated T-25 flask or 6-well plate in Essential 8 medium containing 1× RevitaCell (ThermoFisher catalog#A2644501). Intermediate mesoderm induction was performed by culturing iPSCs in STEMdiff APEL medium (STEMCELL Technologies catalog#5210) or TeSR-E6 medium containing 6-8 μM CHIR99021 (R&D Systems catalog#4423/10) for four days. On Day 4, cells were differentiated in STEMdiff APEL medium or TeSR-E6 medium supplemented with 200 ng/mL FGF9 (R&D Systems catalog#273-F9-025) and 1 μg/mL Heparin (Sigma Aldrich catalog# H4784-250MG).

Manual organoid generation was performed after 7 days of differentiation according to Takasato et al. (Nature Protocols 11, 1681-1692. (2016)) and organoids were cultured for a further 14-18 days prior to harvest.

### Example 2. Bio-Printing Kidney Organoids

### Materials and Methods

Stem cells were prepared as described in Example 1. On Day 7, cells were dissociated with Trypsin EDTA (0.25%, Thermo Fisher catalog#25200-072) or TryPLE Select (ThermoFisher Scientific catalog#12563011). The resulting suspension was counted with a Nexcelom Cellometer to determine the viable cells by trypan exclusion. A single cell suspension of differentiated cells was first counted using a Neubauer hemocytometer (BLAUBRAND catalog# BR7-18605) to obtain cell numbers prior to being centrifuged for 3-5 minutes at 200-300×g to pellet cells in either a 50 mL or 15 mL polypropylene conical tube. After aspirating the supernatant, this cell material was either transferred directly into a 100 uL Gastight syringe (Hamilton Catalog#7656-01) with a 21-25-gauge Removable Needle (Hamilton Catalog#7804-12) for bio-printing, or resuspended to the working cell density with STEMdiff APEL or TESR-E6 media prior to transfer for bio-printing. All syringes containing cellular bio-ink were loaded onto the NovoGen MMX bio-printer, primed to ensure cell material was flowing, and user-defined aliquots of bio-ink were deposited on to 0.4 μm polyester membranes of 6-well (Corning Costar catalog#3450) Transwell permeable supports.

Histological Staining

Kidney organoids were fixed overnight at 4° C. in 2% or 4% paraformaldehyde (Electron Microscopy Sciences, Hatfield, Pa.), pre-embedded in HistoGel (Thermo Fisher, Carlsbad, Calif.), then dehydrated and infiltrated with paraffin using a TissueTek VIP tissue processing system (Sakura Finetek USA, Torrance, Calif.). Planar or transverse 5 μm sections were obtained using a Leica RM 2135 microtome (Leica Biosystems, Buffalo Grove, Ill.). Sections were baked, de-paraffinized and hydrated to water prior to staining following a standard regressive staining protocol using SelecTech staining solutions (Leica Biosystems, Richmond, Ill.; Haematoxylin #3801570, Define #3803590, Blue Buffer #3802915, and Eosin Y 515 #3801615). Stained slides were serially dehydrated, cleared, and mounted in Permaslip (Alban Scientific Inc, St. Louis, Mo. #6530B). Images were acquired on a Zeiss Axio Imager A2 with Zeiss Zen software (Zeiss Microscopy, Thornwood, N.Y.).

Section and Whole Mount Immunofluorescence

For paraffin-embedded organoids, deparaffinized sections were antigen retrieved in citrate buffer, pH 6.0 (Diagnostic BioSystems, Pleasonton, CA #K035) then blocked in 5% chick serum diluted in TBS-T (v/v) prior to immunofluorescence. For whole mount organoids, organoid harvest, fixation and blocking, and immunofluorescence of prepared sections and whole organoids was performed as described previously (Vanslambrouck J M, et al. J Am Soc Nephrol 30, 1811-1823 (2019)). Images were obtained as described in Vanslambrouck J M, et al. or using an Andor spinning disk confocal microscope with Nikon 25×1.05NA silicone immersion objective.

Diameter Measurements

The cross-sectional diameter of the organoids was assessed over time by image-based analysis using ImageJ (version 1.51). Gross images were collected following print on Day 7 at a fixed distance with a 2× objective from plate surface. Each sample was manually outlined using the elliptical selection tool and used to calculate area in pixels for each image. Circular area values were converted to diameter in mm using the following equation:

$$D ( mm ) = \sqrt{\frac{4 \times ( Area ( in pixels ) / ( 78792.49 \frac{{pix}^{2}}{{mm}^{2}} ) )}{π}}$$

Extrusion Bio-Printing Using Dry Paste

During optimisation of extrusion bio-printing, a comparison was made with settled wet paste versus a dry paste generated to recapitulate the packed cell density used when preparing a manual organoid. To achieve a dry paste of this density within the extrusion syringe, the prepared syringe was loaded into a proprietary adaptor to enable centrifugation at 400×g within a 50 mL polypropylene conical tube. Syringe/Adaptor assemblies were centrifuged for a total of 9 minutes to mirror the manual protocol.

### Results

A bio-ink comprising a cell paste was bio-printed with a single point deposition (ratio 0). This single point deposition (or dot) was used to assess whether or not starting density and printing conformation would influence final morphology. Following bio-printing, the single point deposition (ratio 0) tissue forms a domed structure that has similar properties to a manually produced kidney organoid, and as such, the single point deposition (ratio 0) may also be referred to as a bio-printed organoid.

Varied organoid conformations were generated by changing the deposition ratio within the custom software interface, while scaling the organoid length so that each organoid was formed from a constant 1.1×10⁵ cells deposited in a volume of ˜0.55 μl. Starting with a bio-ink comprising a wet cell paste of a set cell density, the same number of cells was bio-printed with deposition being varied, ranging from a line of ˜3 mm (ratio 10) to a line of cells ˜12 mm long (ratio 40). This enabled assessment of whether or not starting density would influence final morphology as detailed below. In each case the inventors varied the line length so that the absolute number of starting cells in each organoid would be approximately equal. Line organoids had a single point deposition (˜10% total) at the start of the pattern to ensure even fluid flow. ‘Dot’ organoids had an equivalent cell volume added to the total so that cell numbers remained matched. During deposition the needle was positioned 300 microns from the Transwell surface. In all cases deposition ratios are based on a 25-gauge needle and 100 μl syringe.

Following bio-printing, the bio-printed organoid is cultured for 1 hour in the presence of 5 to 10 μM CHIR99021 in either STEMdiff™ APEL or TeSR-E6 medium in the basolateral compartment of the Transwell culture plate and subsequently cultured until Day7+5 in STEMdiff™ APEL or TESR-E6 medium supplemented with 200 ng/mL FGF9 and 1 μg/mL Heparin (media only in the basolateral compartment). From Day7+5 to Day 7+18, organoids are grown in STEMdiff™ APEL of TeSR-E6 media medium without supplementation. Kidney organoids can be cultured until harvest from Day 7+12 to Day 7+20. Tissues were maintained under the same conditions as those described above.

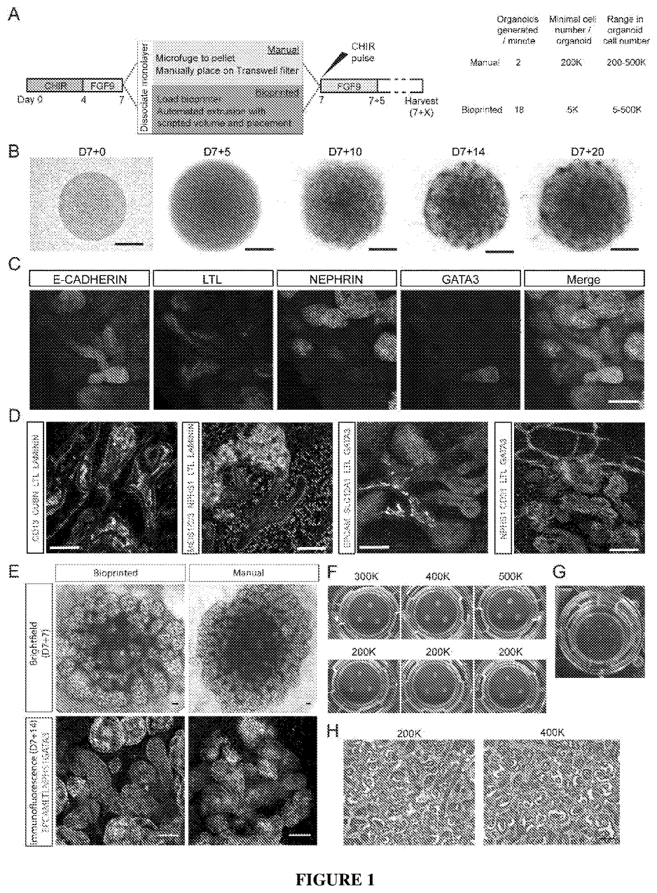

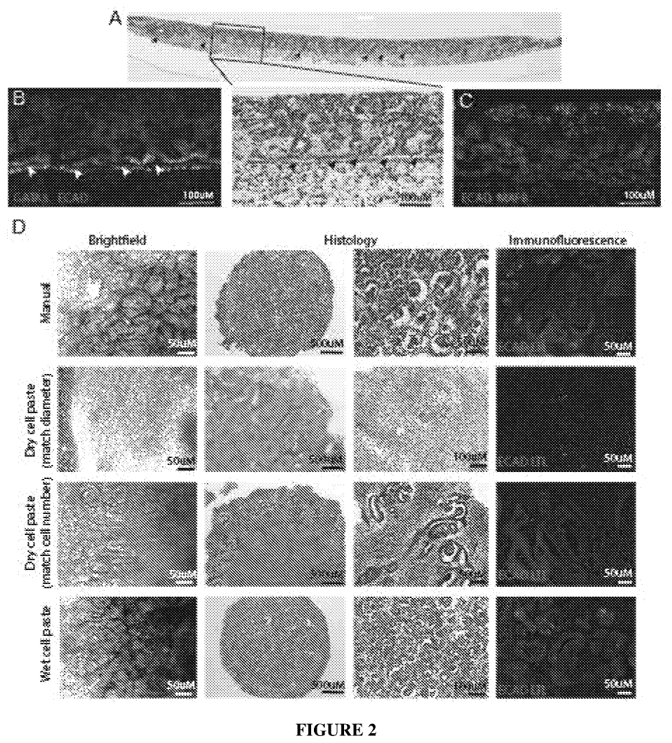

The resulting bio-printed organoids showed spontaneous formation of nephrons across the subsequent 20 days of culture (FIG. 1ABE). Immunofluorescence was used to establish the presence of classically patterned nephrons revealing the presence of podocytes (NEPHRIN), proximal tubules (LTL, CUBN), distal tubules/loop of Henle thick ascending limb (TAL; ECAD, SLC12A1) and connecting/ureteric epithelium (GATA3, ECAD) (FIG. 1CD). The presence of additional cellular components, including endothelial cells (CD31) and renal stroma (MEIS1/2) was also evident (FIG. 1D). Histological sections through bio-printed organoids revealed the presence of a contiguous connecting epithelium (ECAD, GATA3) across the width of the tissue from which individual nephrons radiated (FIG. 2ABC). It should be emphasised that cell paste represents cells only and does not incorporate any associated ECM or hydrogel matrix. The patterning achieved was compared to the outcome when the cell paste was centrifuged to remove all remaining media, creating a packed ‘dry’ cell paste. Subsequent culture of dry paste-derived organoids showed no evidence of nephron formation (FIG. 2D).

To directly compare the cellular complexity of bio-printed with manually-pelleted organoids, the same monolayer differentiation was subjected to both approaches. The resulting kidney organoids were analysed using brightfield imaging and immunofluorescence, demonstrating that bio-printed kidney organoids showed morphological equivalence to manual kidney organoids (FIG. 1E).

### Example 3. Bio-Printed Kidney Organoids with Higher Throughput and Reduced Organoid Size

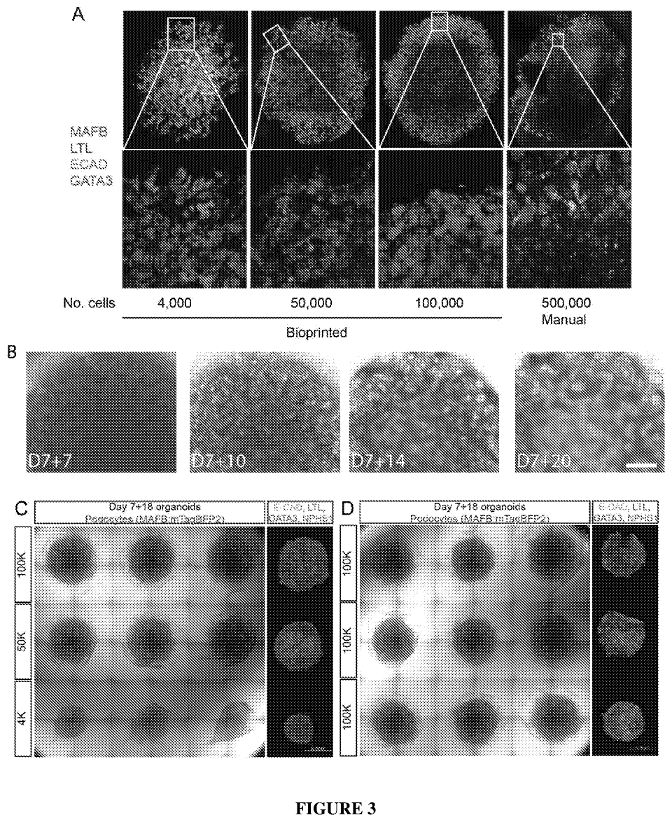

The automated process of bio-printing organoids applied here facilitated the deposition of approximately 1 micromass every 3 seconds, with very high reproducibility of organoid diameter (Table 1). While it is feasible to manually place micromasses consisting of as few as 2×10⁵ cells onto 24-well Transwell plates, bio-printing enabled accurate placement of multiple micromasses into the same filter (3-9 organoids per filter for 6-well plates) (FIG. 1FG). It was also possible to reduce the number of cells used to generate the initial micromass without any loss of histological complexity within the organoid (FIG. 1FH). The yield and throughput of the kidney organoid generation process could therefore be substantially increased, with kidney structure patterning evident in organoids bio-printed from as few as 4×10³ cells (FIG. 3AC). Indeed, the reproducibility of cell paste deposition, as assessed by volume printed and resulting mean diameter, showed a coefficient of variation between 1% and 4% (Table 1).

TABLE 1
Reproducibility of deposition

| Organoid | Volume Bio- | Organoid | Mean Diameter | |
|---|---|---|---|---|
| Size | printed | Number | of Deposit (mm) | % CV |
| 100K | 0.49 μL | 24 | 1.79 | 3.68 |
| 200K | 0.98 μL | 24 | 2.30 | 1.08 |
| 500K | 2.43 μL | 24 | 3.12 | 2.93 |

The Cell line transferability of bio-printing for kidney organoid generation was extensively evaluated using a variety of human induced pluripotent stem cell lines. Both control, reporter and patient-derived iPSC lines successfully generated kidney tissue when bio-printed in this fashion. For example, the use of a specific reporter line in which a blue fluorescent protein has been inserted under the control of the MAFB gene promoter (MAFB^{mTagBFP2}) facilitated the fluorescence imaging of viable tissue to assess relative patterning, including the visualisation of podocyte differentiation in the glomeruli that form at one end of each kidney nephron (FIG. 3B).

### Example 4. Bio-Printed Kidney Organoids for Compound Testing in 96-Well Format

### Materials and Methods

Bio-printed organoids were prepared using the methods outlined in Example 2.

Bio-ink Viability and Concentration Assay

Dispensed bio-ink was sampled before and after the printing of 2 rows (24 organoids) of a 96-well plate. Printed bio-ink was dispensed directly into 1.5 mL Eppendorf tubes filled with APEL medium to dilute and counted using a Nexecelom Cellometer (Nexecelom Biosciences) with trypan blue exclusion. The Nexcelom results were placed into JMP for visualization and statistical analysis. A t-test was performed for analysis with only two conditions compared, a one-way ANOVA and Tukey comparison of means was performed for analysis with more than two conditions compared, and a bivariate fit was performed, using the fit mean, linear fit line, and 95% confidence interval to determine significant trends.

Drug-Induced Nephrotoxicity Studies

Doxorubicin (Sigma-Aldrich, D1515) stock solution was prepared in DMSO. Amikacin, Tobramycin, Gentamycin, Neomycin, and Streptomycin were all procured through Sigma Aldrich (St. Louis, Mo.) and prepared as a 25 mg/ml solution in APEL media. Dosing for 6-well nephrotoxicity studies was performed by initially diluting doxorubicin DMSO stock in APEL media, and subsequently diluting further with additional media to achieve concentrations ranging from 0.3 to 10 μM. Dosing for 96-well nephrotoxicity evaluation was performed by serial dilution. For Doxorubicin, serial dilution of DMSO stocks was added to APEL media to achieve concentrations ranging from 24 nM to 25 μM. Aminoglycoside stock solutions were diluted serially with APEL media to generate dosing concentrations ranging from 1.5 μg/mL to 25 mg/mL. Drug dosing was initiated after day 21 or day 22 of the differentiation protocol. Dosing was performed by applying the full well volume of APEL medium±test article to the apical basket of a Transwell permeable support (4 mL for 6-well plates, 300 μL for 96-well plates). As media containing test articles was added to the Transwell permeable support, the organoids were fully submerged and exposed to any added compounds as the apical and basolateral compartments equilibrated. Drug-supplemented medium was replaced every other day until designated harvest time point.

Organoid Viability Assessment

Kidney organoid viability following drug treatment was assessed by measuring ATP content with CellTiter-Glo or CellTiter-Glo 3D viability assays (Promega, Madison, Wis., USA). In brief, harvested organoids from bio-printed in 6-well plates were individually loaded into Precellys tubes (Bertin Technologies, Bretonneux, France) with CellTiter-Glo buffer and dissociated using a Precellys 24 tissue homogenizer (Bertin Technologies, Bretonneux, France). Homogenized organoids were incubated at room temperature for 10 minutes, then centrifuged at 1000 g for 2 minutes to separate buffer from homogenizing beads. Supernatants were transferred to a white opaque 96-well plate for luminescence measurement on a microplate reader (BMG Labtech, Germany). Presented 6-well viability results are a composite of 3 independent experiments with each normalized to respective control ATP levels within each study. To analyse the ATP content in organoids bio-printed on 96-well plates, all media was aspirated and CellTiter-Glo 3D reagent was added to the apical chamber of Transwell permeable support. The plate was shaken at 400 rpm for 5 minutes at room temperature, and then allowed to sit for 25 minutes prior to luminescent measurement in a white opaque 96-well plate on a microplate reader (BMG Labtech, Germany). Viability analysis was reported as percent of control by normalizing the ATP content of treated organoids relative to control organoids. Fitting of viability results was performed with GraphPad Prism 7.03 software (La Jolla, Calif.) using a four-parameter dose-response curve (Equation 1):

Y=Bottom+(Top−Bottom)/(1+((X^{HillSlope})/(IC₅₀^{HillSlope}))) (Equation 1)

Quantitative RT-PCR Gene Expression Analysis Following Drug Exposure

Total RNA extraction from kidney organoids following drug exposure was performed using an Rneasy Mini kit (Qiagen, Germany) per manufacturer's instructions. RNA was quantified with spectrophotometry with a NanoDrop 2000 (Thermo Fisher, Carlsbad, Calif.). To analyse gene expression, TaqMan Fast One-Step qPCR Master Mix (Applied Biosystems, Foster City, Calif.), TaqMan Probes for genes of interest (ThermoFisher, Carlsbad, Calif.), and house-keeping gene probes (Applied Biosystems, Foster City, Calif.) were combined in assigned wells with RNA. All qPCR reactions were performed and analysed on a StepOnePlus qRT-PCR system (Applied Biosystems, Foster City, Calif.). All data was normalized to house-keeping gene GAPDH prior to normalizing to control samples.

### Results

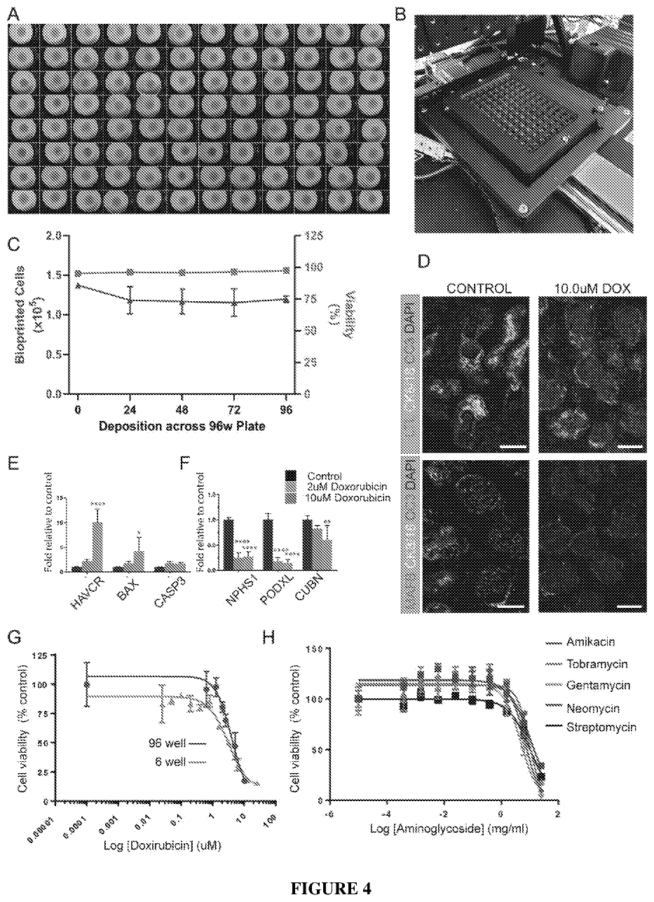

While the kidney plays a crucial role in the elimination of xenobiotics, the uptake of a variety of compounds via tubular specific solute channels places the kidney at risk for nephrotoxic injury. Preclinical screening for nephrotoxicity using primary renal proximal tubule epithelial cells (RPTEC) often fails to accurately predict organ-specific toxicity owing to the rapid dedifferentiation of such cells in 2D culture, losing expression of key transporters and metabolic enzymes. While human kidney organoids have the potential to provide a more accurate and predictive tool for modelling drugs responses, this in part relies upon the capacity to generate large numbers of viable and reproducibly patterned organoids with a low coefficient of variation (cv). To this end, automated bio-printing was further scaled down (1.0×105 starting cells per organoid) and adapted for fabrication of individual organoids onto 96-well Transwell filters (FIG. 4AB). The accuracy of cell count and cell viability was reproducible across all 96 wells with overall cell viability ranging from 93 to 99% (FIG. 4C). As a proof of concept for the application of this approach to nephrotoxicity testing, the effect of administration of a known podocyte toxin, the chemotherapeutic agent Doxorubicin, was first evaluated using bio-printed organoids after treatment for 72 hours in either 2 μM or 10 μM Doxorubicin (FIG. 4D-F). Immunofluorescence staining of resulting organoids showed evidence of specific activation of caspase 3 and loss of MAFB staining within the podocytes of the organoid glomeruli in response to 10 μM Doxorubicin (FIG. 4D). Quantitative RT-PCR (qRT-PCR) showed the upregulation of the kidney injury molecule KIM1 (HAVCR) and the apoptotic indicator, Bcl2-associated X protein (BAX) at 10 μM (FIG. 4E). Doxorubicin also downregulated key podocyte markers NPHS1 and PODXL at 2 μM, while the proximal tubule gene CUBN was only downregulated in response to 10 μM Doxorubicin (FIG. 4F), suggesting differential cell type-specific sensitivity with concentration. To further evaluate dose response, organoids were bio-printed into either 6-well or 96-well format and treated with 24 nM-25 μM Doxorubicin, using ATP content as a viability readout. Viability was affected by Doxorubicin exposure in a dose-dependent fashion with both 6- and 96-well formats producing similar IC50 values in response to treatment (6-well IC50: 3.9±1.8 μM; 96-well IC50: 3.1±1.0 μM) (FIG. 4G). Aminoglycosides are a class of broad-spectrum antibiotics commonly used to treat infections caused by Gram-negative pathogens. Kidney injury due to acute tubular necrosis is a common complication of aminoglycoside therapy due to high intracellular accumulation within proximal tubule cells.

To assess the response of kidney organoids to this class of compound, organoids were bio-printed in a 96-well format and treated with a panel of known nephrotoxic aminoglycosides, including Amikacin, Tobramycin, Gentamycin, Neomycin and Streptomycin, across a wide concentration range. Cell viability as measured by cellular ATP content was decreased in a concentration-dependent fashion following 72-hour treatment with all aminoglycosides evaluated (FIG. 4H).

Bio-printed kidney tissue as exemplified herein thus represents a practical approach to drug testing applicable to assessing the nephrotoxicity of new agents or drug scaffolds with the reproducibility needed to support preclinical safety assessments.

### Example 5. Conformation of Bio-Printed Kidney Tissue Alters Nephron Patterning and Number

As well as providing greater quality control and increased throughput, generating bio-printed organoids using the methods disclosed herein enabled investigation of the effect of changing organoid conformation on tissue morphology. Extrusion bio-printing allows control over the scale and conformation of the cellular micromass formed via precise positioning and movement of the needle tip in 3 dimensions as the cells are extruded.

### Materials and Methods

Bio-printed organoids were prepared using the methods outlined in Example 2.

Bead based analysis of cell density and height at print

Cell paste was spiked with 4 um Tetraspec beads (Thermo-fisher) at 1 ul bead suspension per 50 ul of paste. Organoids were imaged within 2-3 hours of bio-printing to capture brightfield and fluorescent bead signal and again at various times during organoid culture. Imaging was performed using an Andor dragonfly spinning disk confocal with 4×0.2NA Nikon objective, capturing z-stacks beginning at the Transwell surface and continuing until no further bead signal was detected. Fiji (Schindelin, J. et al. Nature Methods, 9, 676-682. (2012)) was used to stitch tiled datasets and generate maximum projections of the bead image. A custom Python script was used to count individual beads in each dataset and final count data was analysed in R. Surface areas derived from bead distributions were used to approximate organoid height at time of print as the height of a shape with vertical sides and the same surface area and volume as the deposited organoid.

Organoid Height Measurements at D7+0

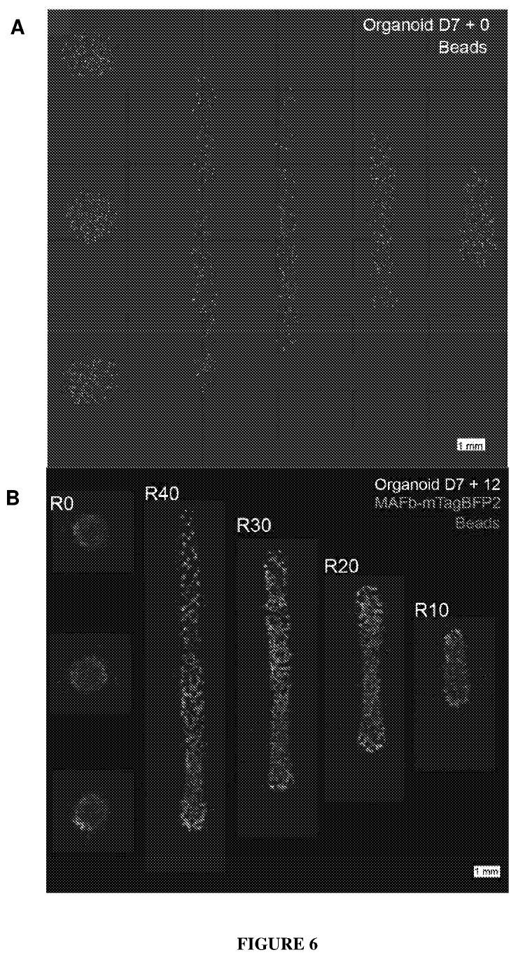

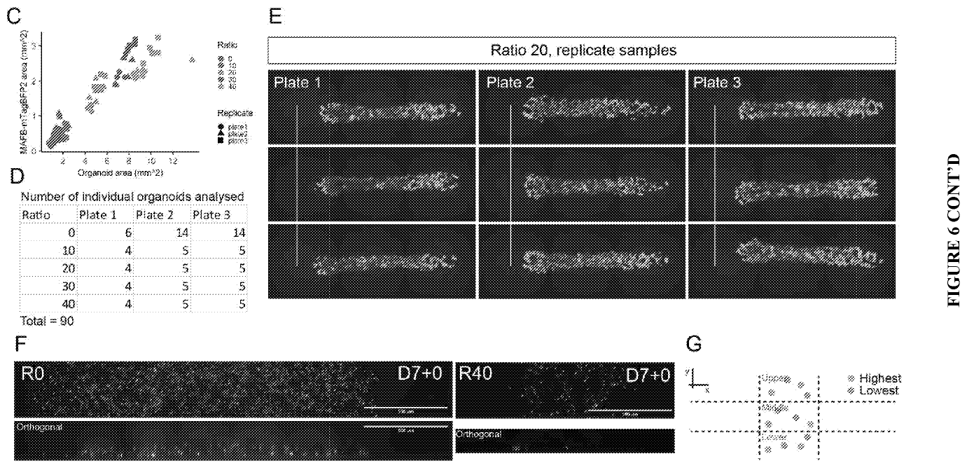

The height of organoids was assessed by image-based quantification of pre-labelled cells using Fiji (Schindelin, J. et al.). Prior to bio-printing 10% of cells were removed and labelled with CellTrace Far Red (ThermoFisher, C34564) according to manufacturer instructions. Labelled cells were mixed back in with the remaining cells and bio-printed to give sparse labelling in the micromass. Two independent sets of organoids were characterised in this way at D7+0 by removing the Transwell containing organoids and placing it flat on a dish (Sarstedt) with a small amount of media. This allowed imaging with a much smaller working distance but prevented the organoids from drying out. Images were captured using an Andor Dragonfly spinning disk with a Nikon 1.15 NA 40× Water immersion objective, capturing images at 0.325×0.325×0.5 micron voxel size. The highest and lowest points of the image stacks were manually measured under the orthogonal view in Fiji. For each sample, the image was equally split into three sections (up, middle & down) in the X-Y plane along the Y-axes (FIG. 6G). Then, in each section, two highest points and two lowest points were recorded in the centre area of the image across the 300 micron range (150 micron from the centre to both −X and +X directions). In general, six highest points and six lowest points were then collected for each condition. The height of the organoids was calculated as:

H(mm)=[Average(6 highest points slides number)−Average(6 lowest points slides number)]×Voxel depth (mm)

Data were compiled in R for analysis and plotting.

Quantitative Imaging of Reporter Cell Lines

Bio-printed D7+12 organoids were live imaged via brightfield and for mTagBFP2 intensity with an Apotome.2 fluorescent microscope (Zeiss). For automated imaging, Transwells were transferred into glass bottomed 6-well dishes (CellVis) and imaged using an Andor Dragonfly spinning disk confocal with a 4×0.2NA Nikon objective. Fiji was used to stitch tiled datasets (Schindelin, J. et al. Nature Methods, 9, 676-682. (2012)). Python scripts using the scikit-image library (Van der Walt, s. et al. PeerJ, 19, 2e453. (2014)) were used to segment and measure the regions of mTagBFP2 signal. The total size of each organoid was approximated by calculating a convex hull around each mTagBFP2 area. Organoid length was approximated by the major axis length of each object. A small number of organoids were excluded from the final analysis based on a ratio of mTagBFP2 positive pixels: total pixels >0.8 that was indicative of segmentation errors that were manually verified.

Bulk-RNAseq Transcriptional Profiling

RNA was extracted from D7+12 organoids using Bioline Isolate II Mini/Micro Kits (Bioline, New South Wales, Australia) as per manufacturer's instructions. RNA was used to generate libraries for sequencing using an Illumina Novoseq 6000 sequencer. Fastq files were trimmed using Trimmomatic (0.35). Mapping to the human genome (GRCh38) was read counting was performed using STAR aligner (2.5.3a) (Dobin, A. et al. Bioinformatics 29, 15-21. (2013)). EdgeR (3.26.5) (Ritchie, M. E., et al.. Nucleic Acids Research. 43, e47 (2015)) was used for library normalization and differential gene expression testing using a quasi-likelihood negative binomial generalized log-linear model.

### Results

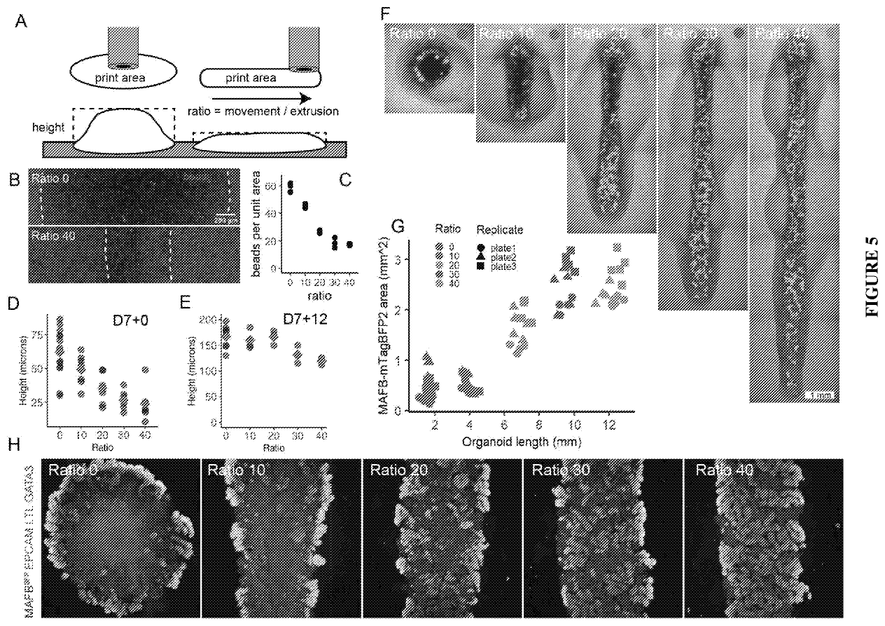

Changing the speed of tip movement for a given rate of cell extrusion allows fine control over tissue height (thickness) as cells are spread, and subsequently aggregate, over larger surface areas (FIG. 5A). Tissue conformations were defined in terms of the deposition ratio, given by the ratio of tip movement along the Transwell surface to the volume of cell suspension deposited. The bio-printer was programmed to create organoids comprising the same total cell number (1.1×10⁵ cells) but varying from a single point deposition (ratio 0, no tip movement at extrusion) to a line of cells ˜12 mm long (ratio 40, movement of 12 mm during extrusion) (FIG. 5 AF). The end result was the formation of a classical organoid structure, where the micromass is deposited as a ‘dot’, to organoids created as ‘lines’ of extruded cell paste. With increasing deposition ratios, the inventors increased the line length to maintain the same absolute number of starting cells in each organoid approximately equal (1.1×10⁵ cells) giving rise to thinner cell masses spread out over a larger surface area. To confirm this empirically, cell paste was spiked with fluorescent beads that would undergo a similar degree of spreading but were easily imaged and automatically quantified at printing (FIG. 5 B, FIG. 6). This allowed the calculation of number of beads per mm² of Transwell surface area occupied (FIG. 5C). As expected, bead density dropped as cells were spread over a greater distance, with approximately three-fold difference between the most and least dense condition (FIG. 5C). The inventors also measured tissue height in the first 24 hours after bio-printing using 3D confocal microscopy. Measuring tissues where cells had been sparsely labelled allowed us to carefully identify the position of cells at the upper and lower limits of each organoid, confirming that higher deposition ratios gave rise to higher tissue masses (FIG. 5 D, FIG. 6F-G).

Replicate sets of organoids at the measured conformations were generated and allowed to differentiate and pattern for 12 days after bio-printing. The absolute tissue height of each organoid after 12 days of culture was measured and compared to the approximated starting height at cell extrusion (FIG. 5 DE). While height increased in both conformations as they grew, the thicker starting organoids remained thicker after culture (FIG. 5E). For these experiments, cell paste was generated using a MAFB^{mTagBFP2} reporter line as described above, enabling efficient imaging of the area of glomerular tissue across replicate live samples (FIG. 5 F, FIG. 6). MAFB^{mTagBFP2} expression coincided with staining for the NPHS1 (nephrin) protein, illustrating the specificity of MAFB-driven blue fluorescence to the podocytes within the forming glomeruli (FIG. 12). Hence, fluorescence imaging of viable organoids enabled the quantification of MAFB-positive area as a surrogate for nephron number. An image processing script was applied to calculate the area of each organoid that contained mTagBFP2-positive structures (MAFB-expressing podocytes of the glomeruli) as a measure of nephron number. Organoids with a long, thin starting conformation had a greater total mTagBFP2-positive glomerular area than small thick organoids (FIG. 5 G), despite being derived from an equal number of starting cells. This trend was consistent across the gradient of densities and was likely due to a larger volume of nephron tissue overall, as all conditions contained glomerular structures. High resolution imaging of individual glomeruli in each conformation confirmed glomerular structures were of a similar size irrespective of organoid conformation (FIG. 12). Hence, thin organoids bio-printed with higher deposition ratio show increased nephron number.

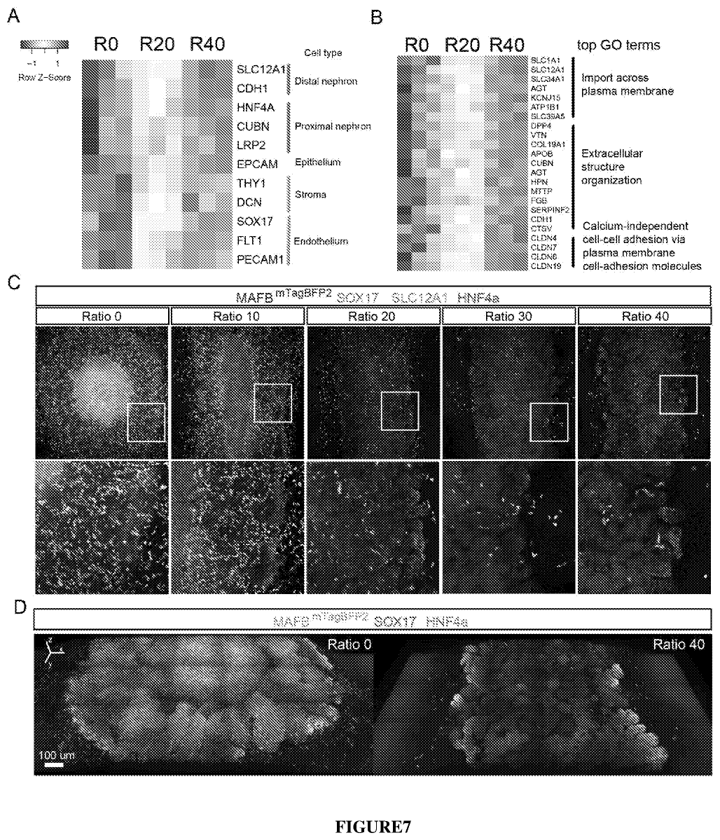

As well as glomeruli number, changes in organoid conformation appeared to affect organoid morphology, with unpatterned stromal tissue most apparent in the centre of ratio 0 organoids (FIG. 5 H). To examine this shift in patterning further, bulk-RNAseq transcriptional profiling was performed to compare ratio 0 ‘dot’ organoids with ‘line’ organoids of two different lengths (ratio 20 and ratio 40), all generated simultaneously and with the same starting cell number. Genes related to epithelial formation (CDH1, EPCAM) and tubule patterning and function (HNF4A, CUBN, LRP2, SLC12A1) were upregulated at ratio 40 (R40), while genes related to vascular (FLT1, SOX17, PECAM) and stromal/fibroblast (THY1, DCN) development were upregulated at ratio 0 (R0) (FIG. 7A,). A GO analysis of pathway changes also suggested improved membrane transport, extracellular 228 organization and cell-cell adhesion in R40 lines compared to bio-printed R0 dots (FIG. 7B). Such changes may reflect changes in relative ratios of cell types or individual levels of gene expression within such cell types. High resolution imaging of ratio 0 and ratio 40 stained organoids showing the location of glomeruli (endogenous mTagBFP2), proximal tubules (HNF4A) and endothelial cells (SOX17) revealed the presence of a wide rim of tissue containing a vascular network in dots that was reduced in lines (FIG. 7D). These conventional micromass dots also showed a clear central core in which nephrons were not forming, as evidenced by non-specific secondary antibody staining (FIG. 7D). Conversely, when organoids were bio-printed as a line, nephrons were present uniformly across the width of the tissue (FIG. 7D).

### Example 6. Single Cell RNAseq Comparison of Cellular Composition and Maturation Between Organoid Conformations

While there is a clear change in nephron uniformity when organoid conformation is altered, significant evidence has previously been identified for variation in patterning between individual organoid differentiation experiments, even when performed with the same cell line. To investigate the reproducibility of this change in morphology and determine whether relative cellular composition or maturation of individual component cell types varies with organoid mode of manufacture (manual versus bio-printed) or conformation (dot versus line) the inventors performed extensive transcriptional profiling (single cell RNA-sequencing; scRNAseq) of three organoid conformations (manual organoids, bio-printed ratio 0 deposition ‘dots’ [R0] and bio-printed ratio 40 deposition ‘lines’ [R40]).

### Materials and Methods

Single Cell RNA Sequencing Library Generation and Analysis

Four replicate organoid sets were generated, where each replicate was derived from an independent pool of D7 differentiated iPSCs derived from 3 monolayer culture wells. For each pool cells were loaded into the bio-printer to print a pattern consisting of 3 R0 ‘dots’ and 3 R4 ‘lines’ per well, over 10-12 wells (2 plates). At the same time the remaining portion of the cell pool was used to generate manual organoids. Bio-printed organoids were generated from 1.1×10⁵ cells each, while manual organoids were generated from 2.3×10⁵ cells, as it was not technically possible to manually manipulate smaller masses. Replicate sets were processed sequentially on the same day so that cells were always loaded and printed within a short period of time. Cells were printed approximately 10 minutes after loading, and the run was complete within ˜20 minutes of loading.

Organoids were dissociated at D7+12 following previously published methods (Vanslambrouck J M, et al. J Am Soc Nephrol 30, 1811-1823 (2019)). For each of R0 and R40, 9 organoids derived from 3 wells (3 per condition, per well) were dissociated. For manual 3 organoids per replicate were dissociated. Replicates were multiplexed following the method of Soeckius et al. (Genome Biol. 19, 224. (2018).). Cells were stained for 20 minutes on ice with 1 μg of BioLegend TotalSeq-A anti-human hashtag oligo antibody (BioLegend TotalSeq-A0251, 0252, 0253, 0254). Cells were washed 3 times then pooled at equal ratios for sequencing. A single library was generated for each suspension/condition (manual, R0, R40), composed of equally sized pools of each replicate (Set 1-4). Libraries were generated following the standard 10× Chromium Next GEM Single Cell 3′ Reagent Kits v3.1 protocol except that ‘superloading’ of the 10× device was performed with ˜30 k cells (Lun, A. T., et al. F1000Research 5, 2122. (2016)). Hash tag oligo (HTO) libraries were generated following the BioLegend manufacturer protocol. Sequencing was performed using an Illumina Novoseq.

10×mRNA libraries were demultiplexed using CellRanger (3.1.0) to generate matrices of UMI counts per cell. HTO libraries were demultiplexed using Cite-seq-count (1.4.3) to generate matrices of HTO counts per cell barcode. All data were loaded into Seurat (3.1.4) and HTO libraries were matched to mRNA libraries. Seurat was used to normalise HTO counts and determine cutoffs to assign HTO identity per cell (cutoff was typically 100-200 counts per cell). Doublet and unassigned cells were removed, as were cells with mitochondrial content greater than 15% or number of genes less than 1000, to obtain filtered datasets with final sizes: manual −9963 cells, R0-8912 cells, R40-13525 cells. Genes were removed that contained counts in less than 20 cells. The combined datasets contained a median of 2034 genes expressed per cell, with a median of 5499 UMI counts per cell.

Data were normalised using the SCTransform method (Lun, A. T., et al. F1000Research 5, 2122. (2016)) and integrated using Seurat to obtain a single dataset. Clustering was performed initially to identify clustering belonging to stroma, nephron, or endothelial compartments. The Clustree package (Wolock S L, et al. Cell Syst, 8: 281-291 (2019)) was used visualise clustering and determine a stable clustering resolution. Nephron and stromal populations were re-normalised with SCTransform and clustered to obtain a finer resolution view of cell heterogeneity. At this level of resolution, the inventors were able to identify clusters with a high computational doublet score, using Scrublet (0.2.1) (Lindstrom, N. O. et al. J Amer Soc Nephrol 29, 806-824. (2018)) and an identity that appeared to combine two known cells types. These were presumed to be unidentifiable doublets consisting of a single HTO ID and were removed from further analysis. Marker analysis was performed using the Seurat FindMarkers function, limited to positive markers (i.e. increased expression within a cluster) above 0.25 log fold-change. Marker lists were exported and cluster identities were determined by comparison with published human single cell data (Chen, J. et al. Nucleic Acids Research 37, W305-311. (2009)) or Gene ontology analysis using ToppFun (Berg S. et al. Nature Methods, 16, 1226-1232. (2019)).

Differential expression testing was performed by summing counts to produce a ‘pseudo-bulk’ count per replicate per cluster using the sumCountsAcrossCells function in Seater (1.12.2), to produce a matrix of gene counts over 12 conditions (4 replicates per organoid conformation). This count matrix was used as input to do differential expression testing in EdgeR (3.26.5) using a quasi-likelihood negative binomial generalized log-linear model implemented in the glmQLFFit function. For differential expression testing within clusters genes appearing as differentially expressed in more than 3 clusters were removed from further analysis, to remove potential batch effects and focus on genes specific to a particular cell type that may be more biologically relevant. Frequently changing genes tended to be mitochondrial and ribosomal genes. Genes were considered differentially expressed if they had an adjusted p value <0.05.

Comparison of Organoids to Human Fetal Kidney Data Using Prediction of Cell Identity

The raw fastq files for the week 11, 13, 16 and 18 single cell datasets published in Hochane et al. 2018 were downloaded from Gene Expression Omnibus and mapped to the reference genome GRCh38-3.0.0 using cellranger. The Seurat package (3.1.5)⁵² was used to perform quality control and analysis. Cells with less than 750 features were removed, the SCTransform method was used to normalise and scale the raw counts then dimensional reduction was performed. The datasets were integrated using the fastMNN method as implemented within the SeuratWrappers package (0.1.0). After an initial clustering the subset identified as nephron was isolated and reanalysed to identify the Progenitors, Pre-Pod, Podocyte, Pre-Tubule, Distal and Proximal cell populations. The Podocyte and Proximal cell populations were further analysed to identify the stages of maturation present within these lineages. The model used to identify the cell types was generated using the scPred package (0.0.0.9) based upon the nephron subsets of the integrated human fetal kidney data as a reference. This produced a model that would classify cells into one of the nephron sub-categories (Progenitors, Pre-Pod, Podocyte, Pre-Tubule, Distal and Proximal). This model was then applied to the organoid single cell datasets to define component cell types.

### Results

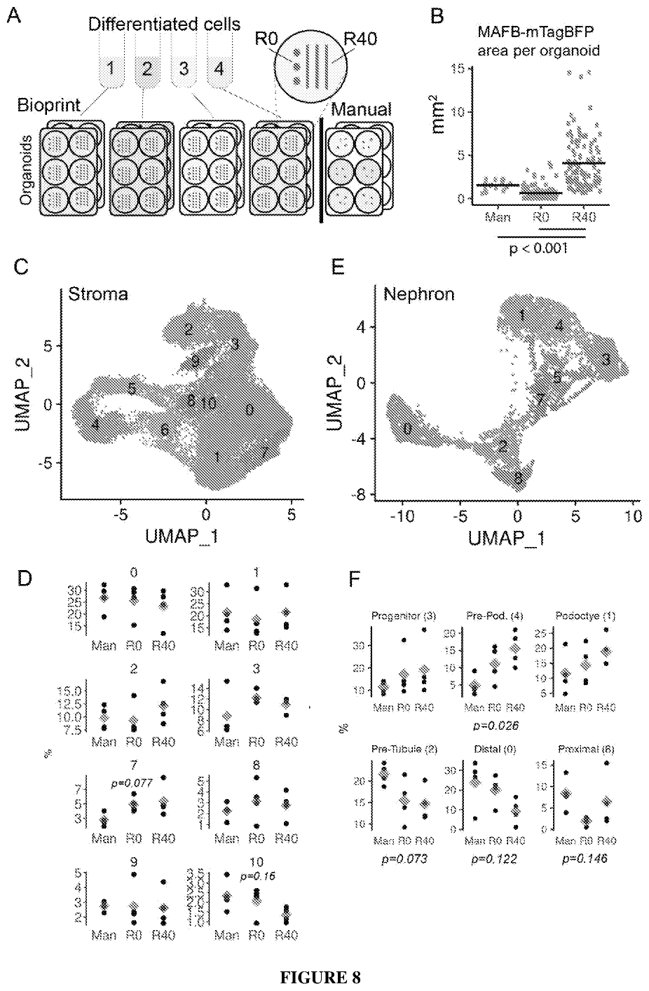

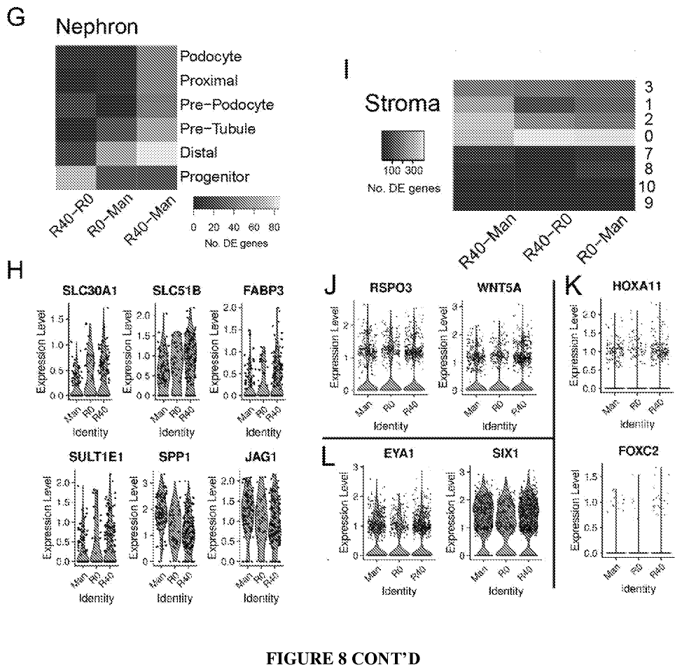

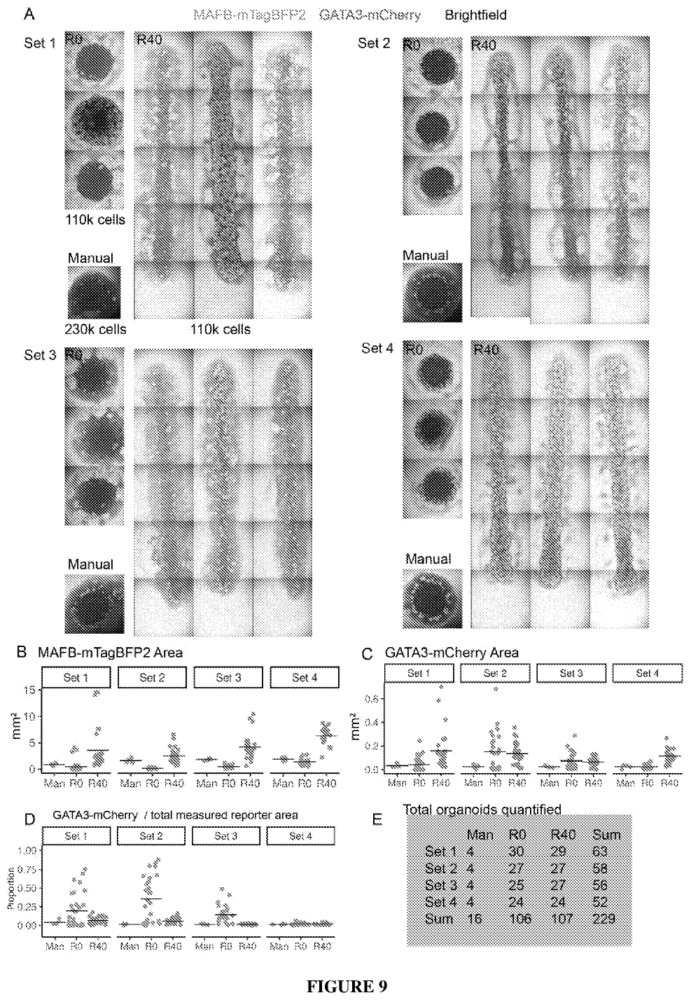

To address experimental variation, libraries were generated from 4 individually barcoded pools of cells representing replicate experiments for each condition, allowing us to robustly assess changes in both population and gene expression between conditions. Each replicate organoid set was generated from a distinct starting pool of differentiated iPSC (MAFB^{mTAGBFP2}-GATA3^{mCherry}) cells and that were bio-printed to produce R0 dots and R40 lines, while manual organoids were made from the same cells in parallel (FIG. 8A). Filtered scRNAseq libraries represented greater than 8000 individual cell transcriptomes per organoid conformation. Quantification of glomerular (MAFB^{mTagBFP2}) and distal nephron (GATA3mcherrY) fluorescence of all organoids generated (n=229 organoids, from 4 replicate sets across 10 plates) confirmed the presence of the previously observed organoid morphology for all conformations, with a clear and quantifiable increase in abundance of nephrons in bio-printed lines, despite the same starting cell number (FIG. 8B, FIG. 9). Bio-printed lines also contained a greater abundance of nephrons compared to manually made organoids which, due to technical limitations mentioned earlier, are made with a larger starting cell number (manual: 2.3×10⁵, R40: 1.1×10⁵, FIG. 8B, FIG. 9).

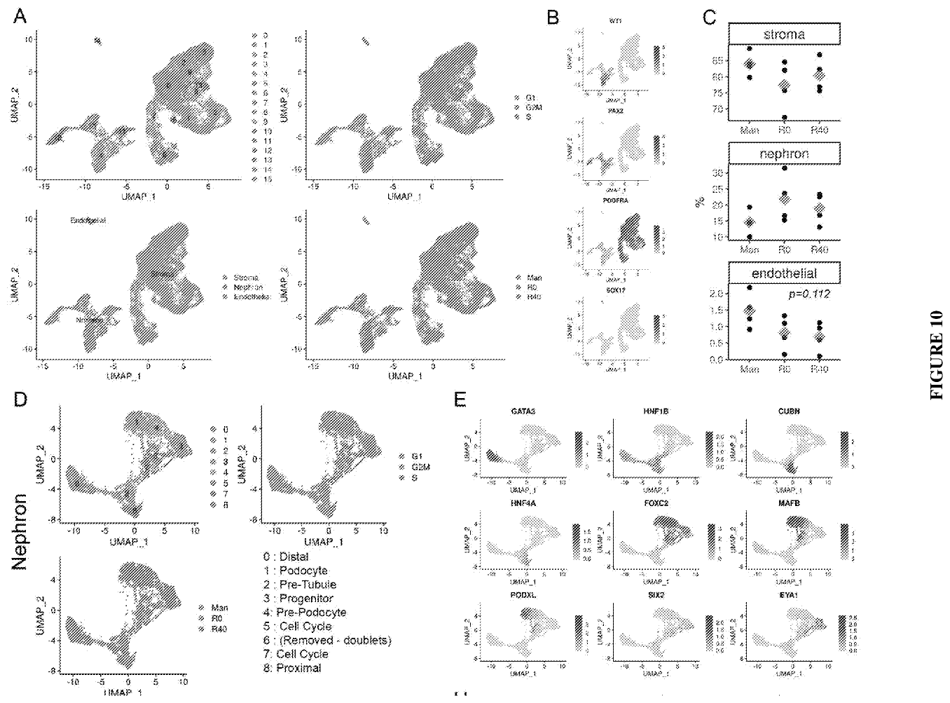

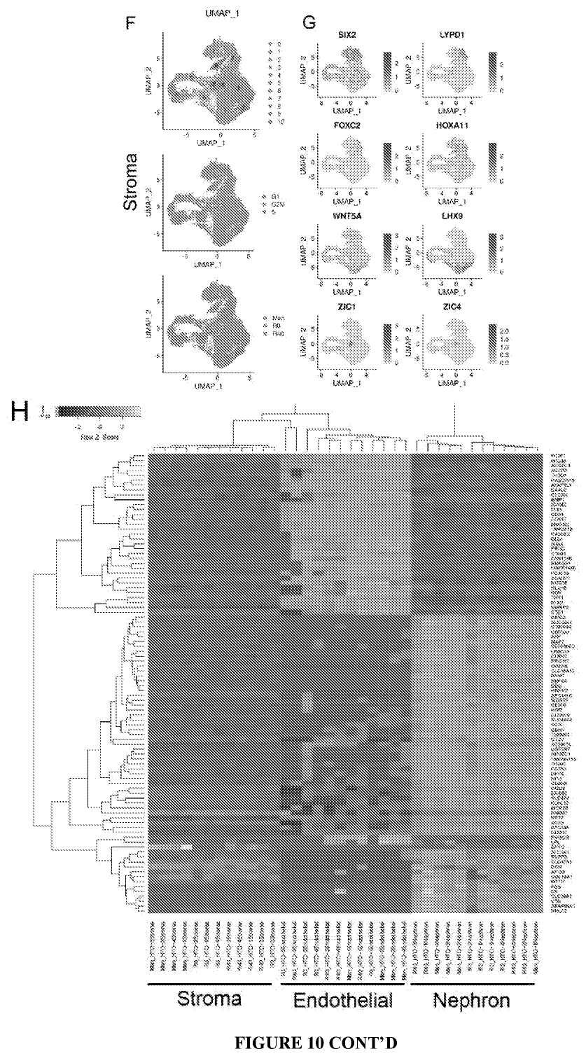

All single cell datasets were integrated using Seurat (Nature Biotechnology. 36, 411-420. (2018)) allowing the broad identification of endothelial, stromal and nephron clusters in all organoid conformations (FIG. 10A-C).

To determine the cell types contributing to the differential gene expression seen in the bulk profiling (FIG. 7), the combined transcriptional profile of each main cell type was used to recreate a ‘pseudo bulk’ expression profile. This confirmed that genes upregulated in bulk RNAseq of R0 dots were markers of endothelial cells, while genes upregulated in R40 lines were nephron markers (FIG. 10H).

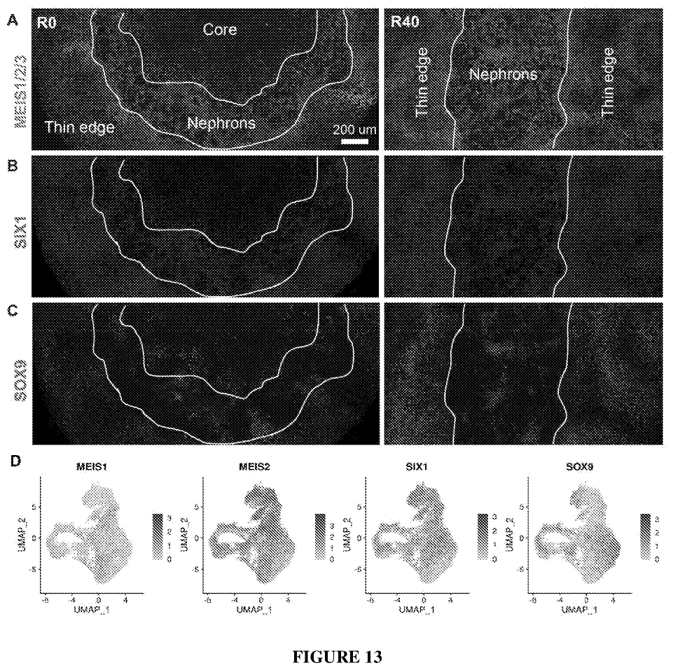

Re-clustering of the stromal cells present within all organoid conformations revealed 10 distinct clusters (FIG. 8C). While there was a trend towards an increase in cluster 7 (expressing WNT5A, LHX9) and a decrease in cluster 10 (expressing ZIC1, ZIC4) in bio-printed organoids, these differences were not statistically significant (FIG. 8D, FIG. 10). Overall, all stromal clusters were present in all organoid conformations with no statistically significant difference in proportion of each cell type. This was surprising given the apparent unpatterned centre in R0 and manual organoids. However, re-analysis of these organoids using immunofluorescence for stromal markers identifying the majority of the dataset (MEIS1/2/3, SIX1 and SOX9) suggested an area of reduced cellularity in this central region (FIG. 13). Hence, the central core was likely a minor contributor to any cell cluster.

Higher resolution re-clustering of nephron lineage cells in the scRNAseq dataset revealed the presence of all major nephron cell types in all organoid conformations (FIG. 8E-F, FIG. 10D-E), with clear expression of MAFB in podocytes, HNF4A in proximal tubule and GATA3 in distal tubule clusters (FIG. 10D-E). There was a significant increase in the prevalence of early podocytes (‘Pre-Pod’) (mean values of ˜5% vs ˜10-15%) (FIG. 8F) and a trend towards increased podocytes (‘Pod’) in bio-printed versus manual organoids, as well as a trend towards increased prevalence of distal tubule in manual and R0 organoids, the latter being supported by an increase in the proportion of GATA3^{mCherry} expressing distal nephron in R0 organoids (FIG. 9D). However, all identified cell clusters were present in all organoid conformations (FIG. 8F, FIG. 10D). The inventors conclude that the patterning is very similar between all organoid conformations, but that the total nephrons formed is greater in bio-printed lines.

### Kidney Organoids Generated as Bio-Printed Lines Show Improved Proximal Tubule Maturation and Increased Nephron Number

To investigate potential differences in maturation, the inventors identified genes within each cell cluster that were significantly differentially expressed between conformations. This revealed the greatest difference between manual organoids and R40 bio-printed lines (FIG. 8G), notably in the identity of the distal nephron. There were less differences between individual nephron cell types between R0 and R40 bio-printed organoids, with the greatest number of differentially expressed genes occurring within the nephron progenitors (FIG. 8G). Importantly, there was evidence of improved maturation of the proximal tubular epithelium in bio-printed R40 lines, but not bio-printed R0 dots, compared to manual organoids. Genes previously associated with mature tubule function and metabolism, including key solute channels (SLC30A1, SLC51B and SULT1E1) and fatty acid metabolism-related gene FABP3 were significantly increased in R40 vs manual organoid proximal tubule cells (FIG. 8H). Conversely, significantly higher expression of markers such as JAG1 and SPP1 in manual organoid cells suggested less maturity (FIG. 8H).

Differential expression analysis within stromal clusters identified the greatest difference between conformations within stromal clusters 0, 1, 2 and 3 (FIG. 8I). Clusters 2 and 3, with identify most similar to early kidney forming mesenchyme, showed a significant upregulation of kidney development genes in bio-printed R40 lines, including HOXA11, FOXC2, EYA1 and SIX1 as well as developmental signalling genes WNT5A and RSPO3 (FIG. 8J-L). Thus, while this stromal cell type was present in all conformations, in bio-printed lines these cells appear to have an identity that more closely resembled early nephron progenitors. This may contribute to the increase in nephrons in bio-printed lines.

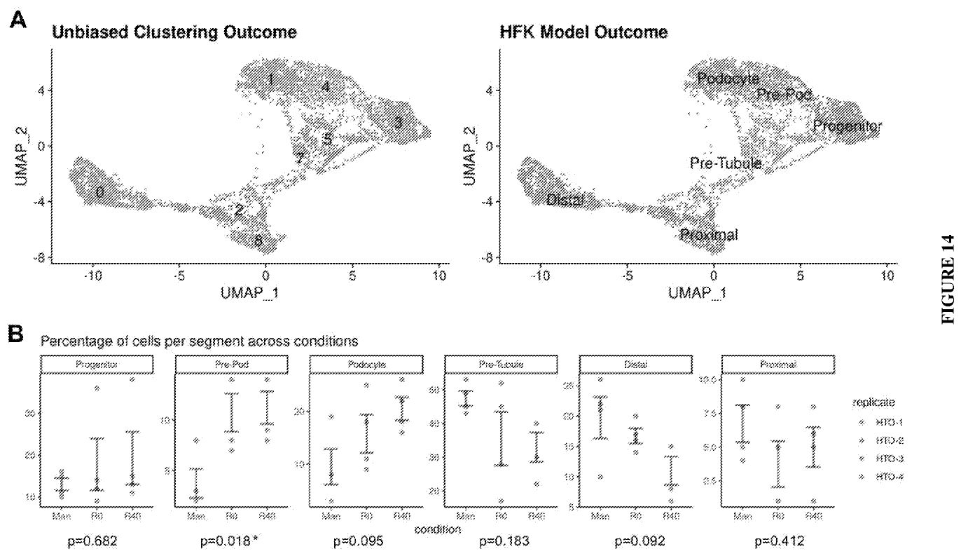

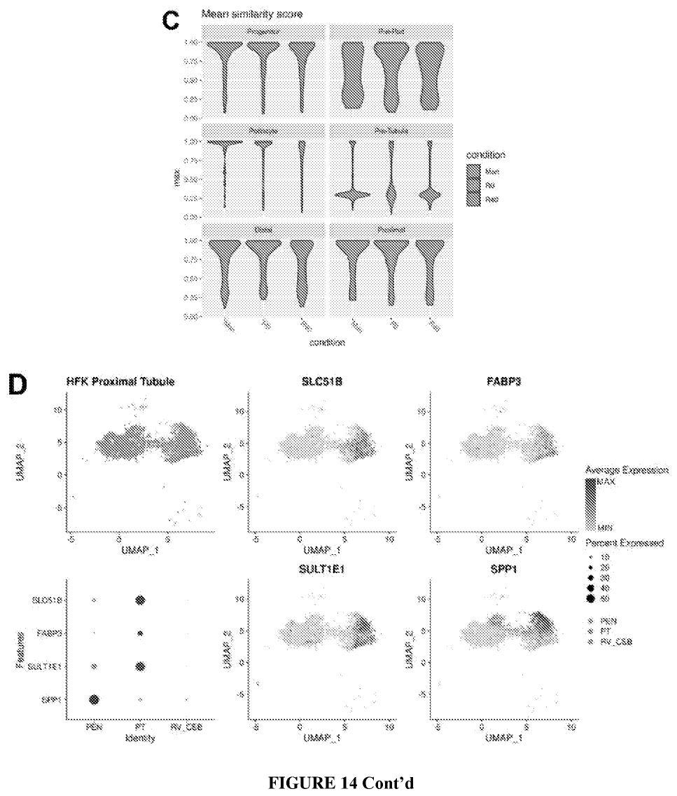

To more definitively compare the maturation of distinct organoid conformations, the inventors used an independent analysis approach in which the cellular identity of each cell within organoids was predicted based upon a direct comparison to human fetal kidney. Using the scPred method the inventors generated a model to predict cellular identity based on transcriptional similarity to a published human fetal kidney (week 11 to 18 gestation) scRNA training dataset (FIG. 14A). This model was used to reanalyse all organoid data to provide an unbiased prediction of cell type within organoids. This approach again identified significant increases in pre-podocyte cells within R40 organoids (FIG. 14B). Genes shown to be differentially expressed in the R40 proximal tubule cell cluster were selectively expressed within the most mature proximal tubule cells in human fetal kidney (FIG. 14D). It can therefore be concluded that, despite experimental variation, bio-printed lines showed improved nephron maturation and increased glomerular number compared to other conformations.

### Example 7. Bio-Printed Kidney Tissue Patches with Increased Nephron Number

The clinical implementation of stem cell-derived kidney tissue requires the capacity to substantially increase the number of nephron structures present in the tissue to be transplanted. Herein the inventors have surprisingly found that changing kidney organoid conformation using extrusion bio-printing it is possible to maximize the final nephron number from a given starting cell number. This suggests that changing conformation may facilitate the generation of larger fields of kidney tissue.

### Materials and Methods

Proximal Tubule Functionality Assay

Functional uptake assays were performed on D7+14 HNF4A^{YFP}-derived patch organoids cultured on 6-well Transwell plates, differentiated and generated as described above. Organoids were incubated (standard 37° C. CO2 incubator conditions) overnight in tetramethylrhodamine isothiocynate-bovine albumin (TRITC-albumin; Sigma-Aldrich) substrate dissolved 1:500 in TeSR-E6 (STEMCELL Technologies) which was added to the basolateral compartment beneath the Transwell insert. Following incubation, organoids were washed in 3 changes of Hank′ Balanced Salt Solution (HBSS; Sigma-Aldrich), transferred to a glass-bottom 6-well plate and live-imaged on a ZEISS LSM 780 confocal microscope (Carl Zeiss, Oberkochen, Germany).

### Results

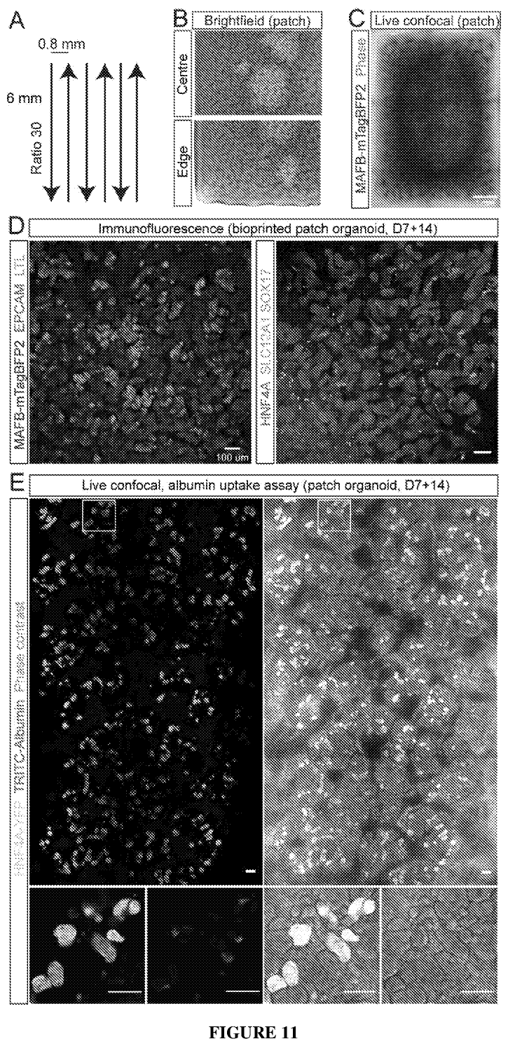

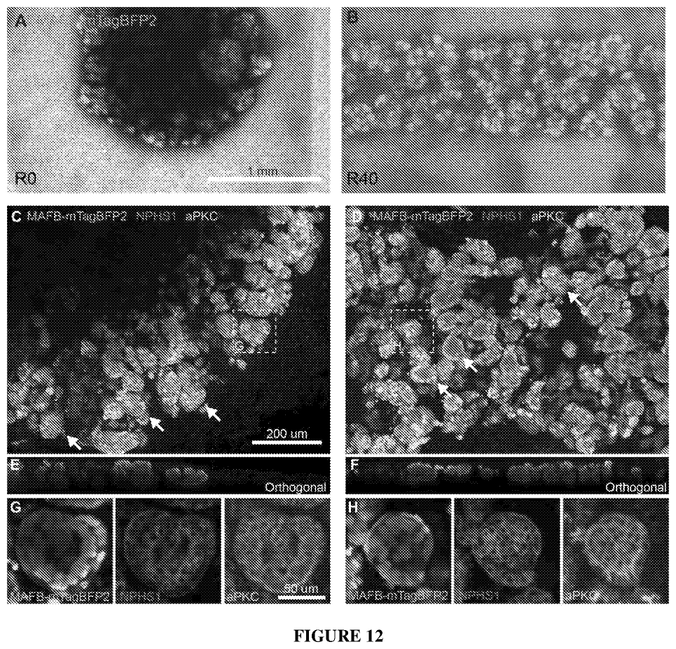

Using the methods described in Examples 2 and 5 and a script to produce a series of parallel lines (FIG. 11A), a bio-printed kidney tissue patch was created extruded using the same extrusion parameters as for the ratio 30 line. In total, the bio-printed kidney tissue patch contained approximately 4×10⁵ cells across a total field of approximately 4.8×6 mm (FIG. 11BC). The resulting kidney tissue patch was examined after 12 and 14 days of culture by brightfield illumination and confocal imaging of an endogenous MAFB^{mTagBFP} reporter signal along with additional kidney markers. These analyses revealed a uniform distribution of epithelial structures and MAFB^{mTagBFP2} expressing glomeruli throughout the patch, as well as the absence central regions lacking nephrons as observed in ratio 0 dot organoids (FIG. 11BC). Patch organoids also demonstrated correctly patterned nephrons, expressing markers of proximal (LTL and HNF4A) and distal tubule/loop of Henle TAL (SLC12A1), surrounded by interstitial endothelial cells expressing SOX17 (FIG. 11D).

A replacement renal tissue must contain nephrons with similar functional capacity to their in vivo counterparts, including glomerular filtration and tubular reabsorption/secretion of water and selected solutes. Given the importance of the proximal tubule for solute reabsorption, patch organoids were generated from a proximal tubule-specific iPSC reporter line in which yellow fluorescent protein (YFP) is inserted under the control of the HNF4A promotor (HNF4A^{YFP} iPS cells). HNF4A^{YFP}-derived bio-printed patches were incubated overnight in a fluorescently tagged protein substrate (TRITC-albumin) that shows affinity for Megalin and Cubilin receptors expressed on podocytes of the glomeruli and proximal tubules. Live confocal imaging revealed specific uptake of TRITC-albumin into YFP-positive proximal tubules, confirming the functionality of these nephron segments (FIG. 11E).

As the relative glomerular number per unit cells extruded was shown to increase by approximately 2.5-5-fold when moving from a set ratio of 0 to 40, it is anticipated that a patch of 4.8×6 mm generated via extrusion of 5×10⁵ cells may contain up to 250-500 nephrons. Hence, a patch of 10×12 mm may generate 1000 nephrons.

Taken together, these data highlight the potential application of patch organoids for the generation of wide fields of functional kidney tissues suitable for bioengineering or screening applications.

### Example 8. Comparative Example Transplantation of Bio-Printed Organoids

Bio-printed kidney organoids (or single point deposition (ratio 0) kidney tissue) as produced in Example 2 were transplanted into mice. Eight-week-old recipient mice (n=8, non-obese diabetic/severe combined immunodeficiency (NOD/SCID), Charles River Laboratories) were anesthetized with isoflurane and injected with temgesic (buprenorphine) for pain relief before surgery. Core body temperature was maintained at 37° C. Via flank incisions, the kidneys were exteriorized, and a small incision was made in the renal capsule. Bio-printed kidney organoids cultured for 7+18 days were bisected and transplanted under renal capsule in the left and right kidney. The mice were anesthetized and sacrificed after 7 and 28 days and the kidneys were collected.

Bio-printed organoids (day 7+18) were fixed in 2% paraformaldehyde (PFA) at 4° C. for 20 minutes. The organoids were permeabilized and blocked in 10% donkey serum in 0.3% TritonX in PBS for 2 hr. Primary antibodies were incubated overnight and were detected by secondary antibodies incubated for 2 hr at room temperature or overnight at 4° C. Organoids under the mouse renal capsule were snap frozen in TissueTek or fixed for 20 min in 2% PFA and stored in PBS for whole mount analysis. Frozen kidney sections (5-10 μm thick) were fixed in 2% PFA for 10 minutes at room temperature and permeabilized in 0.3% TritonX in PBS for 15 minutes. Mouse on Mouse Basic Kit was used to detect structures in the bio-printed kidney organoid and mouse kidney.

Immunofluorescence characterisation of the transplanted and non-transplanted organoids can be performed using antibodies, such as for NPHS1 (AF4269, R&D Systems), WT1 (SC-192, Santa Cruz Biotechnology), CUBILIN (SC20607, Santa Cruz Biotechnology), CD31 (555444, BD Biosciences), ECAD (610181, BD Biosciences), LTL-biotin-conjugated (B-1325, Vector Laboratories), or other examples to highlight organoid-derived tissues or antibodies such as MECA-32 (553849, BD Biosciences), to mark mouse-derived cells types, in this instance mouse endothelium. Live fluorescence imaging can also be used for bio-printed organoids generated using reporter lines.

Transplanted organoids can also be examined using paraffin embedded tissues and sectioned for histological examination after staining using a variety of immunochemical stains, such as haematoxylin and eosin or periodic acid Schiff (PAS) staining. Transplanted organoids could also be examined using transmission or scanning electron microscopy.

The results described herein suggest that bio-printed organoids can be transplanted, remain viable after transplant, draw in a vasculature and show improved maturation. The results here also suggest a capacity to use transplantation assays to compare the relative tubular maturation and success of outcome between bio-printed organoids generated from different starting cell lines, including reporter iPSC lines or patient-derived iPSC lines.

1. Bio-printed kidney tissue, wherein the bio-printed kidney tissue is enriched with nephrons which are distributed throughout the tissue.

2. The bio-printed kidney tissue of claim 1, wherein the bio-printed kidney tissue is a layer of bio-printed tissue comprising a surface area of nephron tissue of greater than 0.2 mm² per 10,000 cells printed.

3. The bio-printed kidney tissue of claim 1 or 2, wherein the bio-printed kidney tissue is a layer of bio-printed kidney tissue comprising about 30,000 cells per mm² or less when printed.

4. The bio-printed kidney tissue of any one of the preceding claims, wherein the bio-printed kidney tissue expresses high levels of any one or more of SULT1E1, SLC30A1, SLC51B, FABP3, HNF4A, CUBN, LRP2, EPCAM and MAFB.

5. The bio-printed kidney tissue of claim 4, wherein the bio-printed kidney tissue comprises nephrons in which the proximal tubule and distal tubule segments express markers of maturation, including HNF4A and SLC12A1.

6. The bio-printed kidney tissue of claim 4 or 5, wherein the bio-printed kidney tissue expresses each of the markers HNF4A, CUBN, LRP2, EPCAM and MAFB.

7. The bio-printed kidney tissue of any one of the preceding claims, wherein the height of the bio-printed kidney tissue is about 50 μm or less when printed.

8. The bio-printed kidney tissue of any one of the preceding claims, wherein the bio-printed kidney tissue has a length of from about 1 mm to about 30 mm and a width of from about 0.5 mm to about 20 mm.

9. The bio-printed kidney tissue of claim 7 or 8, wherein the bio-printed kidney tissue comprises from about 5 to about 100 nephrons/mm² of bio-printed kidney tissue.

10. Bio-printed kidney tissue comprising a predetermined amount of a bio-ink, wherein the bio-ink comprises a plurality of cells, wherein the bio-ink is bio-printed in a layer that is about 50 μm high or less and wherein the bio-printed bio-ink is induced to form kidney tissue.

11. The bio-printed kidney tissue of claim 10, wherein the bio-ink is bio-printed in a layer selected from about 20 μm high to about 40 μm high.

12. The bio-printed kidney tissue of claim 10, wherein the bio-ink is bio-printed in a layer about 30 μm high.

13. The bio-printed kidney tissue of claim 10, wherein the bio-ink is bio-printed in a layer about 25 μm high.

14. The bio-printed kidney tissue of any one of claims 10-14, wherein the predetermined amount of bio-ink comprises between approximately 10,000 cells/μl and approximately 400,000 cells/μl.

15. The bio-printed kidney tissue of any one of claims 10-14, wherein said plurality of cells comprises partly differentiated cells.

16. The bio-printed kidney tissue of any one of claims 10-15, wherein said plurality of cells comprises renal progenitor cells.

17. The bio-printed kidney tissue of claim 16, wherein the renal progenitor cells comprise nephron progenitor cells.

18. The bio-printed kidney tissue of claim 16 or 17, wherein the renal progenitor cells comprise ureteric epithelial progenitor cells.

19. The bio-printed kidney tissue of any one of claims 10-15, wherein said plurality of cells comprises intermediate mesoderm cells.

20. The bio-printed kidney tissue of any one of claims 10-15, wherein said plurality of cells comprises metanephric mesenchyme cells.

21. The bio-printed kidney tissue of any one of claims 10-15, wherein said plurality of cells comprises nephric duct cells.

22. The bio-printed kidney tissue of any one of claims 10-15, wherein said plurality of cells comprises fully differentiated cells.

23. The bio-printed kidney tissue of any one of claims 10-22, wherein said plurality of cells comprises patient-derived cells.

24. The bio-printed kidney tissue of any one of claims 10-23, wherein said plurality of cells comprises cells from a reporter cell line.

25. The bio-printed kidney tissue of any one of claims 10-24, wherein said plurality of cells comprises gene-edited cells.

26. The bio-printed kidney tissue of any one of claims 10-25, wherein said plurality of cells comprises diseased cells, healthy cells, or a combination of diseased and healthy cells.

27. The bio-printed kidney tissue of any one of claims 10-26, wherein the bio-printed kidney tissue comprises a surface area of nephron tissue of greater than 0.2 mm² per 10,000 cells printed.

28. The bio-printed kidney tissue of any one of claims 10-27, wherein the bio-printed kidney tissue comprises about 30,000 cells per mm² or less when printed.

29. The bio-printed kidney tissue of any one of claims 10-28, wherein the bio-printed kidney tissue expresses high levels of any one or more of HNF4A, CUBN, LRP2, EPCAM and MAFB.

30. The bio-printed kidney tissue of claim 29, wherein the bio-printed kidney tissue comprises nephrons in which the proximal tubule and distal tubule segments express markers of maturation, including HNF4A.

31. The bio-printed kidney tissue of claim 29 or 30, wherein the bio-printed kidney tissue expresses each of the markers HNF4A, CUBN, LRP2, EPCAM and MAFB.

32. The bio-printed kidney tissue of any one of claims 1-31, wherein the tissue comprises from about 5 to about 100 nephrons/10,000 cells printed.

33. The bio-printed kidney tissue of any one of claims 10-32, wherein the tissue has an even distribution of nephrons throughout the bio-printed layer.

34. The bio-printed kidney tissue of any one of claims 10-33, wherein the tissue has an even distribution of glomerular structures expressing MAFB throughout the bio-printed layer.

35. The bio-printed kidney tissue of any one of claims 1-34, further comprising a bio-compatible scaffold.

36. The bio-printed kidney tissue of claim 35, wherein bio-ink is bio-printed onto a bio-compatible scaffold.

37. The bio-printed kidney tissue of any one of claim 35 or 36, wherein the biocompatible scaffold is a hydrogel.

38. The bio-printed kidney tissue of any one of claims 35-37, wherein the biocompatible scaffold is biodegradable or bio-absorbable.

39. The bio-printed kidney tissue of any one of claims 10-38, wherein the bio-ink further comprises one or more bioactive agents.

40. The bio-printed kidney tissue of claim 39, wherein said one or more bioactive agents promotes induction of kidney tissue from said plurality of cells.

41. A method for producing bio-printed kidney tissue comprising the steps of: bio-printing a pre-determined amount of a bio-ink onto a surface, wherein the bio-ink comprises a plurality of cells, and wherein the bio-ink is bio-printed in a layer that is about 50 μm high or less; and
inducing the bio-printed, pre-determined amount of the bio-ink to form bio-printed kidney tissue.

42. The method of claim 41, wherein at the step of bio-printing the bio-ink is bio-printed in a layer selected from about 20 μm high to about 40 μm high.

43. The method of claim 41, wherein at the step of bio-printing, wherein the bio-ink is bio-printed in a layer about 30 μm high.

44. The method of claim 41, wherein at the step of bio-printing the bio-ink is bio-printed in a layer about 25 μm high.

45. The method of any one of claims 41-44, wherein the predetermined amount of bio-ink comprises between approximately 10,000 cells/μl and approximately 400,000 cells/μl.

46. The method according to claim 45, wherein the bio-ink comprises about 200,000 cells/μl.

47. The method of any one of claims 41-46, wherein said plurality of cells comprises partly differentiated cells.

48. The method of any one of claims 41-46, wherein said plurality of cells comprises renal progenitor cells.

49. The bio-printed kidney tissue of claim 48, wherein the renal progenitor cells comprise nephron progenitor cells.

50. The method of claim 48 or 49, wherein the renal progenitor cells comprise ureteric epithelial progenitor cells.

51. The method of any one of claims 41-47, wherein said plurality of cells comprises intermediate mesoderm cells, preferably a culture expanded population of stem cell-derived intermediate mesoderm cells.

52. The method of any one of claims 41-47, wherein said plurality of cells comprises metanephric mesenchyme cells.

53. The method of any one of claims 41-47, wherein said plurality of cells comprises nephric duct cells.

54. The method of any one of claims 41-47, wherein said plurality of cells comprises fully differentiated cells.

55. The method of any one of claims 41-54, wherein said plurality of cells comprises patient-derived cells.

56. The method of any one of claims 41-55, wherein said plurality of cells comprises cells from a reporter cell line.

57. The method of any one of claims 41-56, wherein said plurality of cells comprises gene-edited cells.

58. The method of any one of claims 41-57, wherein said plurality of cells comprises diseased cells, healthy cells, or a combination of diseased and healthy cells.

59. The method of any one of claims 41-58, wherein the bio-printed kidney tissue comprises from about 5 to about 100 nephrons/10,000 cells printed.

60. The method of any one of claims 41-59, wherein the bio-printed kidney tissue has an even distribution of nephrons throughout the bio-printed layer.

61. The method of any one of claims 41-60, wherein the bio-printed kidney tissue has an even distribution of glomerular structures expressing MAFB throughout the bio-printed layer.

62. The method of any one of claims 41-61, wherein at the step of bio-printing the bio-ink is bio-printed onto a bio-compatible scaffold.

63. The method of claim 62, wherein the biocompatible scaffold is a hydrogel.

64. The method of any one of claim 62 or 63, wherein the biocompatible scaffold is biodegradable or bio-absorbable.

65. The method of any one of claims 41-64, wherein the bio-ink further comprises one or more bioactive agents.

66. The method of claim 65, wherein said one or more bioactive agents promotes induction of kidney tissue from said plurality of cells.

67. The method of any one of claims 41-66, wherein the step of inducing comprises contacting the bio-printed, predetermined amount of bio-ink with FGF-9.

68. The method of claim 67, wherein the step of inducing comprises contacting the bio-printed, predetermined amount of bio-ink with FGF-9 for a period of 5 days.

69. The method of any one of claims 41-68, wherein the plurality of cells is contacted with a cell culture medium comprising CHIR before being bio-printed.

70. The method of any one of claims 41-69, wherein the bio-printing step uses an extrusion-based bio-printer.

71. The method of any one of claims 41-70, wherein at the step of bio-printing, a dispensing apparatus of a bio-printer is configured to dispense said layer in one or more lines.

72. The method of any one of claims 41-71, wherein at the step of bio-printing, a dispensing apparatus of a bio-printer is configured to dispense said layer in one or more lines so as to form a continuous sheet or patch.

73. Bio-printed kidney tissue produced according to any one of claims 41-72.

74. Bio-printed kidney tissue of any one of claim 1-40, or 73, for use in the treatment of kidney disease or renal failure in a subject in need thereof.

75. Use of bio-printed kidney tissue of any one of claim 1-40, or 73, in the manufacture of a medicament for the treatment of kidney disease in a subject in need thereof.

76. A method of treating kidney disease or renal failure in a subject in thereof, comprising administering to the subject bio-printed kidney tissue of any one of claim 1-40, or 73.

77. The bio-printed kidney tissue of any one of claim 1-40, or 73, for use according to claim 74, the use of claim 75, or the method of claim 76, wherein in said treatment the bio-printed kidney tissue is transplanted under the renal capsule of said subject.
